(19) United States
(12) Patent Application Publication
(10) Pub. No.: US 2005/0222124 A1
(43) Pub. Date: Oct. 6, 2005

(54) Benzenesulfonamide derivatives as antipsychotic agents

(76) Inventors: Steven Mark Bromidge, Verona (IT); David Gwyn Cooper, Harlow (GB); Ian Thomson Forbes, Harlow (GB); Andrew Derrick Gribble, Harlow (GB); Christopher Norbert Johnson, Harlow (GB); Andrew Plightfoot, Harlow (GB); Stephen Frederick Moss, Harlow (GB); Andrew H Payne, Harlow (GB); Shahzad Sharooq Rahman, Harlow (GB); David R. Witty, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/504,111

(22) PCT Filed: Feb. 13, 2003
(86) PCT No.: PCT/EP03/01545

§ 371 Date: Mar. 15, 2005

(30) Foreign Application Priority Data
Feb. 13, 2002 (GB) 0203438.7
Feb. 13, 2002 (GB) 0203437.9
Feb. 28, 2002 (GB) 0204784.3
Feb. 28, 2002 (GB) 0204758.7
May 30, 2002 (GB) 0212548.2
Aug. 23, 2002 (GB) 0219711.9
Oct. 21, 2002 (GB) 0224466.3

(51) Int. Cl. A61K 31/55; C07D 223/16

(52) U.S. Cl. 514/217.02; 540/595

(57) ABSTRACT

The invention provides compounds of formula (I) 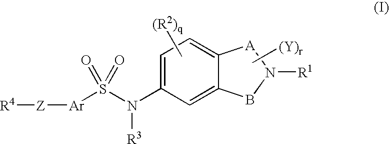 wherein
A and B represent the groups —(CH₂)ₘ- and —(CH₂)ₙ-respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆alkoxyC₁₋₆alkyl, C₃₋₇cycloalkylC₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, C₁₋₆alkylsulfonyloxy, C₁₋₆alkylsulfonylC₁₋₆alkyl, —CO₂C₁₋₆alkyl, —CO₂NR⁷R⁸, —SO₂NR⁷R⁸, C₁₋₆alkylsulfonamido, C₁₋₆alkylsulfonamidoC₁₋₆alkyl, —(CH₂)ₚNR⁷R⁸, C₁₋₆alkylamidoC₁₋₆alkyl, —(CH₂)ₚNR⁷COR⁸, arylsulfonyl, arylsulfonyloxy, arylsulfonylC₁₋₆alkyl, arylsulfonamido, arylcarboxamido, arylsulfonamidoC₁₋₆alkyl, arylcarboxamidoC₁₋₆alkyl, aroyl, aroylC₁₋₆alkyl, arylC₁₋₆alkanoyl, —SO₂NR⁷R⁸, optionally substituted aryl, optionally substituted heteroaryl or optionally substituted heterocyclyl, or a group CONR⁷R⁸ or SO₂NR⁷R⁸ wherein R⁷ and R⁸ together may be fused to form a 5- 7-membered aromatic or non-aromatic heterocyclic ring optionally interrupted by an O or S atom;
R³ represents hydrogen or C₁₋₆alkyl; Ar represents optionally substituted phenyl or optionally substituted monocyclic heteroaryl group;
R⁴ represents optionally substituted aryl or optionally substituted heteroaryl;
R⁷ and R⁸ each independently represent hydrogen, C₁₋₆alkyl or together form a 5- to 7-membered heterocyclic ring;
Z represents a bond, an oxygen atom or C₁₋₆alkyl;
Y represents hydrogen or C₁₋₆alkyl; m and n independently represent an integer selected from 1 and 2; p independently represents an integer selected from 0, 1, 2 and 3; q represents an integer from 1 to 3; r represents an integer from 1 to 4; or a pharmaceutically acceptable salt or solvate thereof. The compounds are useful in therapy, in particular as antipsychotic agents.

This invention relates to novel compounds, pharmaceutical compositions containing them and their use in therapy, in particular as antipsychotic agents.

WO 98/27081, WO 99/02502, WO 99/37623, WO 99/42465 and WO 01/32646 (SmithKline Beecham plc) disclose a series of aryl sulfonamide and sulfoxide compounds that are said to be 5-HT₆ receptor antagonists and which are claimed to be useful in the treatment of various CNS disorders.

WO 01/62737 discloses amino pyrazole derivatives useful for the treatment of obesity and other disorders associated with the NPY receptor subtype Y5.

EP0937723 discloses sulfonamide compounds useful in the treatment of thrombolytic disorders.

WO 01/85695 discloses tetrahydroisoquinoline analogues useful as growth hormone secretagogues.

U.S. Pat. No. 5,684,195 discloses a method of preparing sulfonamides from sulfones.

WO 02/46164 discloses aryl sulfonamide compounds that are said to be useful as selective ER-β ligands in the treatment or prophylaxis of Alzheimer's disease, anxiety disorders, depressive disorders, osteoporosis, cardiovascular disease, rheumatoid arthritis or prostate cancer.

A structurally novel class of compounds has now been found which are useful as antipsychotic agents and for the treatment of other disorders.

According to the invention, there is provided a compound of formula (I): 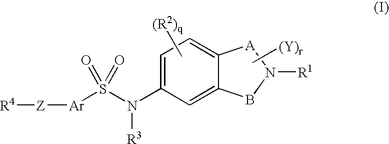
wherein

A and B represent the groups —(CH₂)ₘ- and —(CH₂)ₙ-respectively;

R¹ represents hydrogen or C₁₋₆alkyl;

R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆alkoxyC₁₋₆alkyl, C₃₋₇cycloalkylC₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, C₁₋₆alkylsulfonyloxy, C₁₋₆alkylsulfonylC₁₋₆alkyl, —CO₂C₁₋₆alkyl, —CO₂NR⁷R⁸, —SO₂NR⁷R⁸, C₁₋₆alkylsulfonamido, C₁₋₆alkylsulfonamidoC₁₋₆alkyl, —(CH₂)ₚNR⁷R⁸, C₁₋₆alkylamidoC₁₋₆alkyl, —(CH₂)ₚNR⁷COR⁸, arylsulfonyl, arylsulfonyloxy, arylsulfonylC₁₋₆alkyl, arylsulfonamido, arylcarboxamido, arylsulfonamidoC₁₋₆alkyl, arylcarboxamidoC₁₋₆alkyl, aroyl, aroylC₁₋₆alkyl, arylC₁₋₆alkanoyl, —SO₂NR⁷R⁸, optionally substituted aryl, optionally substituted heteroaryl or optionally substituted heterocyclyl, or a group CONR⁷R⁸ or SO₂NR⁷R⁸ wherein R⁷ and R⁸ together may be fused to form a 5- 7-membered aromatic or non-aromatic heterocyclic ring optionally interrupted by an O or S atom;

R³ represents hydrogen or C₁₋₆alkyl;

Ar represents optionally substituted phenyl or optionally substituted monocyclic heteroaryl group;

R⁴ represents optionally substituted aryl or optionally substituted heteroaryl;

R⁷ and R⁸ each independently represent hydrogen, C₁₋₆alkyl or together form a 5- to 7-membered heterocyclic ring;

Z represents a bond, an oxygen atom or C₁₋₆alkylene;

Y represents hydrogen or C₁₋₆alkyl;

m and n independently represent an integer selected from 1 and 2;

p independently represents an integer selected from 0, 1, 2 and 3;

q represents an integer from 1 to 3;

r represents an integer from 1 to 4;

or a pharmaceutically acceptable salt or solvate thereof.

As a further aspect of the invention, there is provided a compound of formula (I) wherein A, B, Y, Z, q, r, Ar and R¹ to R⁴ have any of the meanings as hereinbefore described, with the proviso that when R¹ represents C₁₋₆alkyl and Y represents hydrogen, Ar cannot represent an optionally substituted monocyclic heteroaryl group.

As used herein, the term “alkyl”, either alone or as part of another group, refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy or hexyloxy.

As used herein, the term “cycloalkyl” refers to a non-aromatic hydrocarbon ring containing the specified number of carbon atoms. For example, C₃₋₇cycloalkyl means a non-aromatic ring containing at least three, and at most seven, ring carbon atoms. Examples of “cycloalkyl” as used herein include, but are not limited to, cyclopropyl, cyclobutyl, cyclopentyl, cyclohexyl and cycloheptyl. A C₆₋₇cycloalkyl group is preferred.

As used herein, the term “halogen” refers to the elements fluorine, chlorine, bromine and iodine. Preferred halogens are fluorine, chlorine and bromine.

As used herein, the term “aryl” refers to a phenyl or a naphthyl ring.

As used herein, the term “heteroaryl” refers to a 5- or 6-membered heterocyclic aromatic ring or a fused bicyclic heterocyclic ring system.

As used herein, the term “heterocyclyl” refers to a 3- to 7-membered monocyclic saturated ring containing at least one heteroatom independently selected from oxygen, nitrogen and sulfur. Examples of suitable heterocyclic rings include, but are not limited to, piperidine and morpholine.

As used herein, the term “5- or 6-membered heterocyclic aromatic ring” refers to a monocyclic unsaturated ring containing at least one heteroatom independently selected from oxygen, nitrogen and sulfur. Examples of suitable 5- and 6-membered heterocyclic aromatic rings include, but are not limited to, furyl, thienyl, pyrrolyl, oxazolyl, thiazolyl, imidazolyl, oxadiazolyl, thiadiazolyl, pyridyl, triazolyl, triazinyl, pyridazyl, pyrimidinyl, pyrazolyl, isothiazolyl and isoxazolyl.

As used herein, the term “fused bicyclic heterocyclic ring system” refers to a ring system comprising two 5- to 7-membered saturated or unsaturated rings, the ring system containing at least one heteroatom independently selected from oxygen, nitrogen and sulfur. Preferably, each ring has 5 or 6 ring atoms. Examples of suitable fused bicyclic rings include, but are not limited to, indolyl, indolinyl, benzofuranyl, benzothienyl, quinolyl, isoquinolyl, tetrahydroquinolyl, benzodioxanyl, indanyl and tetrahydronapthyl.

As used herein, the term “optionally substituted” refers to optional substitution with the named substituent or substituents, multiple degrees of substitution being allowed unless otherwise stated.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include water, methanol, ethanol and acetic acid. Most preferably the solvent used is water and the solvate may also be referred to as a hydrate.

It will be appreciated that for use in medicine the salts of formula (I) should be pharmaceutically acceptable. Suitable pharmaceutically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Other non-pharmaceutically acceptable salts e.g. oxalates, may be used, for example in the isolation of compounds of formula (I) and are included within the scope of this invention. Also included within the scope of the invention are solvates and hydrates of the compounds of formula (I).

Certain of the compounds of formula (I) may form acid addition salts with one or more equivalents of the acid. The present invention includes within its scope all possible stoichiometric and non-stoichiometric forms thereof.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

The groups R², R⁵ and R⁶ may be located on any free position on their respective phenyl rings. The Y group(s) may be located on any free position on the respective ring.

When R², R⁴, R⁵ or R⁶ represent optionally substituted aryl or optionally substituted heteroaryl or R² additionally represents optionally substituted heterocyclyl, the optional substituents may be independently selected from C₁₋₆alkyl, C₁₋₆alkoxy, halogen, trifluoromethyl, trifluoromethoxy, cyano, nitro, —NR⁷R⁸, —C₁₋₆alkylS and —S—C₁₋₆alkyl. More preferably, the optional substituents for the groups R², R⁴, R⁵ and R⁶ are independently selected from chloro, fluoro, bromo, methyl, ethyl, t-butyl, methoxy, trifluoromethyl, trifluoromethoxy, cyano, nitro, —S-methyl, methyl-S and —NR⁷R⁸.

When Ar represents optionally substituted phenyl or optionally substituted monocyclic heteroaryl, the optional susbtituents are independently selected from hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆alkoxyC₁₋₆alkyl, C₃₋₇cycloallylC₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, —C₁₋₆alkylS, C₁₋₆alkylsulfonyloxy, C₁₋₆alkylsulfonylC₁₋₆alkyl, —CO₂C₁₋₆alkyl, —CO₂NR⁷R⁸, —SO₂NR⁷R⁸, C₁₋₆alkylsulfonamido, C₁₋₆alkylsulfonamidoC₁₋₆alkyl, —(CH₂)ₚNR⁷R⁸, C₁₋₆alkylamidoC₁₋₆alkyl, —(CH₂)ₚNR⁷COR⁸, aryl sulfonyl, arylsulfonyloxy, arylsulfonylC₁₋₆alkyl, arylsulfonamido, arylcarboxamido, arylsulfonamidoC₁₋₆alkyl, arylcarboxamidoC₁₋₆alkyl, aroyl, aroylC₁₋₆alkyl, arylC₁₋₆alkanoyl, —SO₂NR⁷R⁸, optionally substituted aryl or optionally substituted heteroaryl, or a group CONR⁷R⁸ or SO₂NR⁷R⁸ wherein R⁷ and R⁸ together may be fused to form a 5- to 7-membered aromatic or non-aromatic heterocyclic ring optionally interrupted by an O or S atom.

Preferably, R¹ represents hydrogen or C₁₋₄alkyl. More preferably, R¹ represents hydrogen, methyl, ethyl, n-propyl, isopropyl, t-butyl or n-butyl. Even more preferably, R¹ represents hydrogen, methyl, ethyl, n-propyl or isopropyl. Even more preferably, R¹ represents hydrogen or methyl.

Preferably, R² represents hydrogen, halogen, C₁₋₆alkyl, C₁₋₆alkoxy, —C₁₋₆alkylS, —S—C₁₋₆alkyl, —NR⁷R⁸ or optionally substituted heterocyclyl. In particular, R² represents methyl, ethyl, methoxy, ethoxy, isopropoxy, bromo, chloro, dimethylamino, —S-ethyl, -ethyl-S or piperidyl. More preferably, R² represents hydrogen, halogen, C₁₋₆alkyl or C₁₋₆alkoxy. Even more preferably, R² represents hydrogen, halogen, C₁₋₄alkyl or C₁₋₄alkoxy. Even more preferably, R² represents hydrogen, dimethylamino, methoxy, ethoxy or isopropoxy.

Preferably, R³ represents hydrogen or C₁₋₄alkyl. More preferably, R³ represents hydrogen, methyl, ethyl, n-propyl or isopropyl. Even more preferably, R³ represents hydrogen, methyl or isopropyl.

Preferably, R⁴ represents phenyl, naphthyl, thienyl, benzofuranyl, furyl, benzothienyl, pyridyl, isoxazolyl and pyrrolyl, all of which may be optionally substituted. More preferably, R⁴ represents phenyl, naphthyl, thienyl, benzofuranyl, furyl or benzothienyl, all of which may be optionally substituted. Even more preferably, R⁴ represents phenyl or thienyl (e.g. 2-thienyl or 3-thienyl).

If R⁴ is optionally substituted, preferably R⁴ is mono- or di-substituted. In particular, when R⁴ is phenyl, the optional substituents may be independently selected from chloro (e.g. 2-, 3- or 4-chloro), bromo (e.g. 4-bromo), fluoro (e.g. 2-, 3- or 4-fluoro), dichloro (e.g. 2,4- or 3,4-dichloro), difluoro (e.g. 2,4-, 3,4- or 3,5-difluoro), trifluoromethyl (e.g. 4-trifluoromethyl), methyl (e.g. 2-, 3- or 4-methyl), t-butyl (e.g. 4-t-butyl), methoxy (e.g. 4-methoxy), trifluoromethoxy (e.g. 4-trifluoromethoxy), cyano (e.g. 4-cyano), nitro (e.g. 4-nitro), dimethylamino (e.g. 4-dimethylamino), -methyl-S (e.g. 4-methyl-S), or methyl and chloro together (e.g. 2-methyl-4-chloro or 3-methyl-4-chloro). More preferably, when R⁴ is phenyl, one of the optional substituents is located at the 4-position relative to the attachment of R⁴ to the rest of the molecule.

When R⁴ is thienyl, the optional substituents may be independently selected from chloro (e.g. 5-chloro) or methyl (e.g. 4- or 5-methyl).

Preferably, R⁷ and R⁸ independently represent hydrogen or C₁₋₄alkyl. More preferably, R⁷ and R⁸ independently represent hydrogen or methyl.

Preferably, Ar represents optionally substituted phenyl.

Preferably, Z represents a bond or oxygen. More preferably, Z represents a bond.

Preferably, Y represents hydrogen.

Preferably, p represents 0.

Preferably, q represents 1.

Preferably, r represents 1.

According to a further aspect of the invention, there is provided a compound of formula (I) wherein Ar represents a phenyl ring, i.e. a compound of formula (IA): 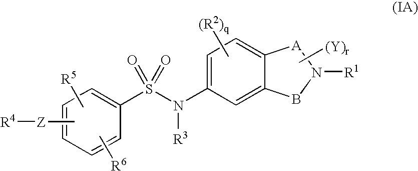
or a pharmaceutically acceptable salt or solvate thereof wherein the groups A, B, R¹ to R⁴, Z, Y, q and r have any of the meanings as given hereinbefore and R⁵ and R⁶ each independently represent hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆alkoxyC₁₋₆alkyl, C₃₋₇cycloalkylC₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, —C₁₋₆alkylS, C₁₋₆alkylsulfonyloxy, C₁₋₆alkylsulfonylC₁₋₆alkyl, —CO₂C₁₋₆alkyl, —CO₂NR⁷R⁸, —SO₂NR⁷R⁸, C₁₋₆alkylsulfonamido, C₁₋₆alkylsulfonamidoC₁₋₆alkyl, —(CH₂)ₚNR⁷R⁸, C₁₋₆alkylamidoC₁₋₆alkyl, —(CH₂)ₚNR⁷COR⁸, aryl sulfonyl, arylsulfonyloxy, arylsulfonylC₁₋₆alkyl, arylsulfonamido, arylcarboxamido, arylsulfonamidoC₁₋₆alkyl, arylcarboxamidoC₁₋₆alkyl, aroyl, aroylC₁₋₆alkyl, arylC₁₋₆alkanoyl, —SO₂NR⁷R⁸, optionally substituted aryl or optionally substituted heteroaryl, or a group CONR⁷R⁸ or SO₂NR⁷R⁸ wherein R⁷ and R⁸ together may be fused to form a 5- to 7-membered aromatic or non-aromatic heterocyclic ring optionally interrupted by an O or S atom.

Preferably, R⁵ and R⁶ independently represent hydrogen, methyl, fluoro or chloro.

According to a further aspect of the invention, there is provided a compound of formula (IA) wherein q represents 1, r represents 1 and Y represents hydrogen, i.e. a compound of the formula (IB): 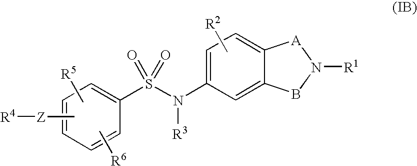
or a pharmaceutically acceptable salt or solvate thereof wherein the groups A, B, R¹ to R⁶ and Z have any of the meanings as given hereinbefore.

According to a further aspect of the invention, there is provided a compound of formula (IB) wherein the R² group is located at the para-position relative to the group B, i.e. a compound of formula (IC): 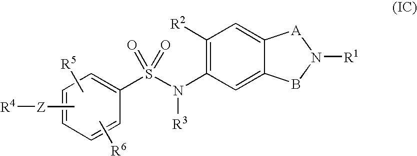
or a pharmaceutically acceptable salt or solvate thereof wherein the groups A, B, R¹ to R⁶ and Z have any of the meanings as given hereinbefore.

According to a further aspect of the invention, there is provided a compound of formula (IB) wherein the group -Z-R⁴ is located at the para-position relative to the sulfonamide group, i.e. a compound of formula (ID)) 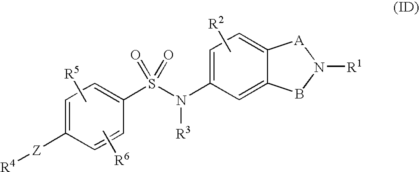
wherein

A and B represent the groups —(CH₂)ₘ- and —(CH₂)ₙ-respectively;

R¹ represents hydrogen or C₁₋₆alkyl;

R² represents hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl, a fused bicyclic heterocyclic ring system or optionally substituted heterocyclyl;

R³ represents hydrogen or C₁₋₆alkyl;

R⁴ represents optionally substituted aryl or optionally substituted heteroaryl;

R⁵ and R⁶ each independently represent hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or a fused bicyclic heterocyclic ring system;

R⁷ and R⁸ each independently represent hydrogen or C₁₋₆alkyl;

Z represents a bond, an oxygen atom or C₁₋₆alkylene;

m and n independently represent an integer selected from 1 and 2;

p independently represents an integer selected from 0, 1, 2 and 3;

or a pharmaceutically acceptable salt or solvate thereof.

According to a further aspect of the invention, there is provided a compound of formula (ID) wherein m is 1 and n is 1, i.e. a compound of formula (IE): 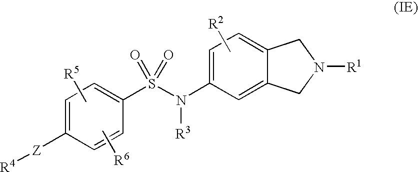
or a pharmaceutically acceptable salt or solvate thereof wherein the groups Z and R¹ to R⁶ have any of the meanings as given hereinbefore.

According to a further aspect of the invention, there is provided a compound of formula (ID) wherein m is 2 and n is 1, i.e. a compound of formula (IF): 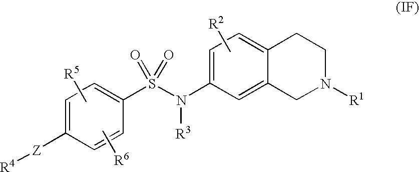
or a pharmaceutically acceptable salt or solvate thereof wherein the groups Z and R¹ to R⁶ have any of the meanings as given hereinbefore.

According to a further aspect of the invention, there is provided a compound of formula (ID) wherein m is 1 and n is 2, i.e. a compound of formula (IG): 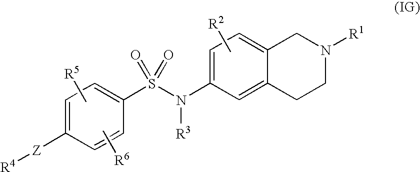
or a pharmaceutically acceptable salt or solvate thereof wherein the groups Z and R¹ to R⁶ have any of the meanings as given hereinbefore.

According to a further aspect of the invention, there is provided a compound of formula (IB) wherein m is 2 and n is 2, i.e. a compound of formula (IH): 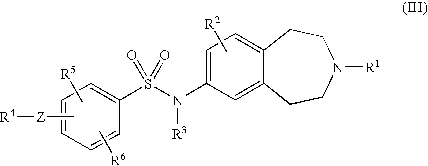
or a pharmaceutically acceptable salt or solvate thereof wherein the groups Z and R¹ to R⁶ have any of the meanings as given hereinbefore.

According to a further aspect of the invention, there is provided a compound of formula (ID) wherein m is 2 and n is 2, i.e. a compound of formula (IJ): 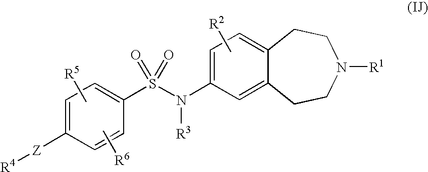
or a pharmaceutically acceptable salt or solvate thereof wherein the groups Z and R¹ to R⁶ have any of the meanings as given hereinbefore.

According to a further aspect of the invention, there is provided a compound of formula (IJ) wherein the R² group is located at the para-position relative to the group B, i.e. a compound of formula (IK): 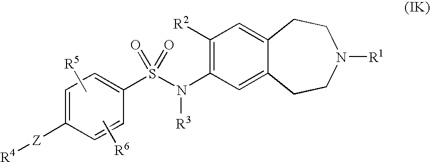
or a pharmaceutically acceptable salt or solvate thereof wherein the groups Z and R¹ to R⁶ have any of the meanings as given hereinbefore.

According to a further aspect of the invention, there is provided a compound of formula (I) wherein R¹ and R³ both represent hydrogen, m and n both represent 2 and Z represents a bond, i.e. a compound of formula (IL): 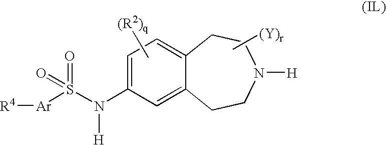
wherein:

R² represents hydrogen, halogen, hydroxy, cyano, nitro, trifluoromethyl, trifluoromethoxy, C₁₋₆alkyl, trifluoromethanesulfonyloxy, pentafluoroethyl, C₁₋₆alkoxy, arylC₁₋₆alkoxy, C₁₋₆alkylthio, C₁₋₆alkoxyC₁₋₆alkyl, C₃₋₇cycloalkylC₁₋₆alkoxy, C₁₋₆alkanoyl, C₁₋₆alkoxycarbonyl, C₁₋₆alkylsulfonyl, C₁₋₆alkylsulfinyl, C₁₋₆alkylsulfonyloxy, C₁₋₆alkylsulfonylC₁₋₆alkyl, arylsulfonyl, arylsulfonyloxy, arylsulfonylC₁₋₆alkyl, C₁₋₆alkylsulfonamido, C₁₋₆alkylamido, C₁₋₆alkylsulfonamidoC₁₋₆alkyl, C₁₋₆alkylamidoC₁₋₆alkyl, arylsulfonamido, arylcarboxamido, arylsulfonamidoC₁₋₆alkyl, arylcarboxamidoC₁₋₆alkyl, aroyl, aroylC₁₋₆alkyl, arylC₁₋₆alkanoyl, or a group CONR⁷R⁸ or SO₂NR⁷R⁸, wherein R⁷ and R⁸ independently represent hydrogen or C₁₋₆alkyl or together may be fused to form a 5- to 7-membered aromatic or non-aromatic heterocyclic ring optionally interrupted by an O or S atom;

Y represents hydrogen or C₁₋₆alkyl;

q represents an integer from 1 to 3;

r represents an integer from 1 to 4;

Ar and R⁴ independently represent phenyl or a monocyclic heteroaryl group each of which may be optionally substituted;

Ar and R⁴ may be optionally substituted by one or more substituents which may be the same or different, and which are selected from those defined for R²;

or solvates thereof.

According to a further aspect of the invention, there is provided a compound of formula (I) or a pharmaceutically acceptable salt or solvate thereof wherein the groups A, B, R¹ to R⁴, Y, q and r have any of the meanings as given hereinbefore and Z represents oxygen or C₁₋₆alkylene.

According to a further aspect of the invention, there is provided a compound of formula (IA) or a pharmaceutically acceptable salt or solvate thereof wherein the groups A, B, R¹ to R⁴, Y, q and r have any of the meanings as given hereinbefore and Z represents oxygen or C₁₋₆alkylene.

According to a further aspect of the invention, there is provided a compound of formula (IB) or a pharmaceutically acceptable salt or solvate thereof wherein the groups A, B and R¹ to R⁶ have any of the meanings as given hereinbefore and Z represents oxygen or C₁₋₆alkylene.

According to a further aspect of the invention, there is provided a compound of formula (IC) or a pharmaceutically acceptable salt or solvate thereof wherein the groups A, B and R¹ to R⁶ have any of the meanings as given hereinbefore and Z represents oxygen or C₁₋₆alkylene.

According to a further aspect of the invention, there is provided a compound of formula (ID) or a pharmaceutically acceptable salt or solvate thereof wherein the groups A, B and R¹ to R⁶ have any of the meanings as given hereinbefore and Z represents oxygen or C₁₋₆alkylene.

According to a further aspect of the invention, there is provided a compound of formula (IE) or a pharmaceutically acceptable salt or solvate thereof wherein the groups R¹ to R⁶ have any of the meanings as given hereinbefore and Z represents oxygen or C₁₋₆alkylene.

According to a further aspect of the invention, there is provided a compound of formula (IF) or a pharmaceutically acceptable salt or solvate thereof wherein the groups R¹ to R⁶ have any of the meanings as given hereinbefore and Z represents oxygen or C₁₋₆alkylene.

According to a further aspect of the invention, there is provided a compound of formula (IG) or a pharmaceutically acceptable salt or solvate thereof wherein the groups R¹ to R⁶ have any of the meanings as given hereinbefore and Z represents oxygen or C₁₋₆alkylene.

According to a further aspect of the invention, there is provided a compound of formula (IH) or a pharmaceutically acceptable salt or solvate thereof wherein the groups R¹ to R⁶ have any of the meanings as given hereinbefore and Z represents oxygen or C₁₋₆alkylene.

According to a further aspect of the invention, there is provided a compound of formula (IJ) or a pharmaceutically acceptable salt or solvate thereof wherein the groups R¹ to R⁶ have any of the meanings as given hereinbefore and Z represents oxygen or C₁₋₆alkylene.

According to a further aspect of the invention, there is provided a compound of formula (IK) or a pharmaceutically acceptable salt or solvate thereof wherein the groups R¹ to R⁶ have any of the meanings as given hereinbefore and Z represents oxygen or C₁₋₆alkylene.

According to a further aspect of the invention, there is provided a compound of formula (IL) or a pharmaceutically acceptable salt or solvate thereof wherein the groups R¹ to R⁶ have any of the meanings as given hereinbefore and Z represents oxygen or C₁₋₆alkylene.

In a preferred aspect of the invention, compounds of formula (I) are of the formulae (IE), (IF), (IH), (IJ) and (IK) or a pharmaceutically acceptable salt or solvate thereof wherein the groups Z and R¹ to R⁶ have any of the meanings as given hereinbefore.

Particular compounds according to the invention include those incorporated in Tables 1 to 3 and those specifically exemplified and named hereinafter including, without limitation:

4-(4-Chloro-phenyl)-N-(2,3,4,5-tetrahydro-1H-3-benzazepin-7-yl)-benzenesulfonamide;

4-(4-Chloro-phenyl)-N-(3-methyl-2,3,4,5-tetrahydro-1H-3-benzazepin-7-yl)-benzenesulfonamide;

4-(4-Chloro-phenyl)-N-methyl-N-(2,3,4,5 -tetrahydro-1H-3-benzazepin-7-yl)-benzenesulfonamide;

4-(4-Chloro-phenyl)-N-methyl-N-(3-methyl-2,3,4,5-tetrahydro-1H-3-benzazepin-7-yl)-benzenesulfonamide;

4-(3,4-Dichloro-phenyl)-N-(2,3,4,5-tetrahydro-1H-3-benzazepin-7-yl)-benzenesulfonamide hydrochloride;

4-(4-Chloro-phenyl)-N-(8-methoxy-2,3,4,5-tetrahydro-1H-3-benzazepin-7-yl)-benzenesulfonamide hydrochloride;

4-(4-Chloro-phenyl)-N-(8-methoxy-3-methyl-2,3,4,5-tetrahydro-1H-3-benzazepin-7-yl)-benzenesulfonamide hydrochloride;

4-(4-Chloro-phenyl)-N-(1,2,3,4-tetrahydro-isoquinolin-7-yl)-benzenesulfonamide;

4-(4-Chloro-phenyl)-N-(2,3-dihydro-1H-isoindol-5-yl)-benzenesulfonamide hydrochloride;

4-(4-Chloro-phenyl)-N-(2-methyl-2,3-dihydro-1H-isoindol-5-yl)-benzenesulfonamide;

4-(4-Chloro-phenyl)-3-methyl-N-(2,3,4,5-tetrahydro-1H-3-benzazepin-7-yl)-benzenesulfonamide hydrochloride;

4-(4-Chloro-phenyl)-3-methyl-N-(3-methyl-2,3,4,5-tetrahydro-1H-3-benzazepin-7-yl)-benzenesulfonamide;

4-(4-Chloro-phenyl)-3-methyl-N-(8-methoxy-2,3,4,5-tetrahydro-1H-3-benzazepin-7-yl)-benzenesulfonamide hydrochloride;

4-(4-Chloro-phenyl)-3-methyl-N-(8-methoxy-3-methyl-2,3,4,5-tetrahydro-1H-3-benzazepin-7-yl)-benzenesulfonamide;

4-(5-Chloro-thiophen-2-yl)-N-(8-methoxy-3-methyl-2,3,4,5-tetrahydro-1H-benzo[d]azepin-7-yl)-benzenesulfonamide;

4-(5-Chloro-thiophen-2-yl)-2-fluoro-N-(8-methoxy-3-methyl-2,3,4,5-tetrahydro-1H-benzo[d]azepin-7-yl)-benzenesulfonamide;

4-(4-Chloro-phenyl)-N-(8-dimethylamino-3-methyl-2,3,4,5-tetrahydro-1H-benzazepin-7-yl)-benzenesulfonamide hydrochloride and

4-(4-fluorobenzyl)-N-(3-methyl-2,3,4,5-tetrahydro-1H-benzo[d]azepin-7-yl)-benzenesulfonamide hydrochloride.

The compounds of the present invention may be in the form of their free base or pharmaceutically acceptable salts thereof, particularly the monohydrochloride salt.

The present invention also provides a general process (A) for preparing compounds of formula (I) which process comprises:

reacting a compound of formula (II) 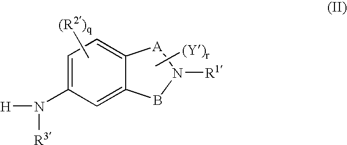
with a compound of formula (III) 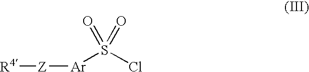
wherein A, B, Z, q and r are as hereinbefore defined and R^{1′}-R^{4′} and Y′ represent R¹ to R⁴ and Y as hereinbefore defined or are groups that may be readily convertible to R¹ to R⁴.

This general method (A) can be conveniently performed by mixing the two components in a suitable solvent such as pyridine or dichloromethane (in the presence of a base), at 0° C.

According to a further aspect of the invention, when compounds of the formula (ID) are prepared by method (A), a compound of formula (II) as hereinbefore defined is reacted with a compound of formula (IIIa) 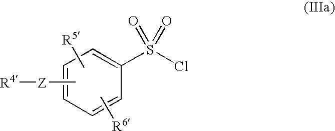
wherein A, B, Z, q and r are as hereinbefore defined and R^{1′}-R^{6′} and Y′ represent R¹ to R⁶ and Y as hereinbefore defined or are groups that may be readily convertible to R¹ to R⁶.

The present invention also provides a general process (B) for preparing compounds of formula (I) wherein Z is a bond, which process comprises:

reacting a compound of formula (IV) 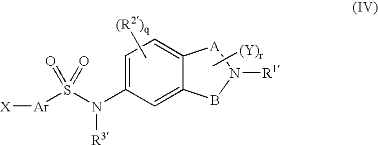
wherein X is a leaving group, such as iodo, bromo or triflate, and A, B, q, r and Y are as hereinbefore defined and R^{1′}—R^{3′} represent R¹ to R³ as hereinbefore defined or are groups that may be readily convertible to R¹ to R³,

with an aryl boronic acid of formula (V) 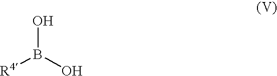
wherein R^{4′} represents R⁴ as hereinbefore defined or is a group that may be readily convertible to R⁴, under standard Suzuki conditions, e.g. treatment of compound (IV) with 4-chlorobenzeneboronic acid in toluene containing aqueous sodium carbonate and a catalytic amount of Pd(PPh₃)₄, at reflux under argon.

According to a further aspect of the invention, when compounds of the formula (ID) are prepared by method (B), a compound of formula (IVa) 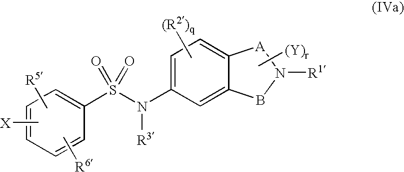
wherein X is a leaving group, such as iodo, bromo or triflate, and A, B, q, r and Y are as hereinbefore defined and R^{1′}—R^{6′} represent R¹ to R⁶ as hereinbefore defined or are groups that may be readily convertible to R¹ to R⁶,

with an aryl boronic acid of formula (V) as hereinbefore defined.

The present invention also provides a general process (C) for preparing compounds of formula (I) which process comprises:

converting a compound of formula (I) 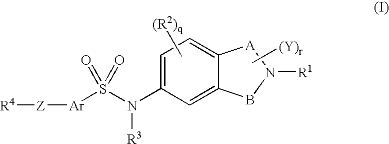
wherein A, B, Z, Y, q, r and R¹ to R⁴ are as hereinbefore defined, into another compound of formula (I) by substituting the group R¹ or the group R³ using conventional techniques.

Interconversion of one of the R^{1′} to R^{4′} groups to the corresponding R¹ to R⁴ groups typically arises when one compound of formula (I) is used as the immediate precursor of another compound of formula (I), or when it is easier to introduce a more complex or reactive substituent at the end of a synthetic sequence.

For example, conversion of R^{1′} from a t-butoxycarbonyl (BOC) group to hydrogen is conducted by the treatment of the N-BOC protected compound with hydrogen chloride in ethanol or dioxan at room temperature.

Conversion of R^{1′} from hydrogen to an alkyl group is conducted by the treatment of the NH compound with the appropriate aldehyde in dichloroethane in the presence of a reducing agent, such as sodium triacetoxyborohydride, or by the treatment of the NH compound with the appropriate alkyl halide, such as iodomethane, under standard alkylation conditions (potassium carbonate in DMF at 60° C.).

Conversion of R^{3′} from hydrogen to an alkyl group is conducted by the treatment of the sulfonamide NH compound with the appropriate alcohol, such as methanol, under Mitsunobu conditions i.e. treatment with diisopropyl azodicarboxylate/triphenylphosphine and methanol in tetrahydrofuran at room temperature.

Compounds of formula (II) are known in the literature or may be prepared by known processes, for example, reduction of the corresponding nitro compound as disclosed in WO 99/14197, or by procedures analogous to these procedures. Suitable examples of an R^{1′} protecting group are trifluoroacetyl or the t-butoxycarbonyl (BOC) group.

Compounds of formula (III) are commercially available or may be prepared by established procedures, for example chlorosulfonylation of a suitable substituted aromatic precursor, using chlorosulfonic acid, for example as described in J. Med. Chem., 2000, 43, 156-166.

Compounds of formula (IV) may be prepared from compounds of formula (II) by the treatment with the appropriate 4-substituted benzenesulfonyl chloride using standard conditions, for example in pyridine or dichloromethane in the presence of a base such as triethylamine at room temperature.

Compounds of formula (V) are commercially available or may be prepared by known methodology, for example lithiation of a suitable substituted bromobenzene at low temperature followed by quenching with tri-isopropylborate and acidic hydrolysis of the reaction product.

Compounds of formula (I) have been found to exhibit affinity for dopamine receptors, in particular the D₃ and D₂ receptors, and are useful in the treatment of disease states which require modulation of such receptors, such as psychotic conditions. Many of the compounds of formula (I) have also been found to have greater affinity for dopamine D₃ than for D₂ receptors. The therapeutic effect of currently available antipsychotic agents (neuroleptics) is generally believed to be exerted via blockade of D₂ receptors; however this mechanism is also thought to be responsible for undesirable extrapyramidal side effects (eps) associated with many neuroleptic agents. Without wishing to be bound by theory, it has been suggested that blockade of the dopamine D₃ receptor may give rise to beneficial antipsychotic activity without significant eps. (see for example Sokoloff et al, Nature, 1990; 347: 146-151; and Schwartz et al, Clinical Neuropharmacology, Vol 16, No. 4, 295-314, 1993). Additionally, certain compounds of formula (I) have antagonist affinity for the serotonin 5-HT_{2A}, 5-HT_{2C} and 5-HT₆ receptors. These additional properties may give rise to enhanced anti-psychotic activity (e.g. improved effects on cognitive dysfunction) and/or reduced eps. These could include, but are not limited to, attenuation of cognitive symptoms via 5-HT₆ receptor blockade (see Reavill, C. and Rogers, D. C., 2001, Investigational Drugs 2, 104-109), and reduced anxiety (see for example Kennett et al., Neuropharmacology 1997 April-May; 36 (4-5): 609-20), protection against eps (Reavill et al., Brit. J. Pharmacol., 1999; 126: 572-574) and antidepressant activity (Bristow et al., Neuropharmacology 39:2000; 1222-1236) via 5-HT_{2C} receptor blockade.

Compounds of formula (I) may also exhibit affinity for other receptors not mentioned above, resulting in beneficial antipyschotic activity.

The compounds of formula (I) are of use as antipsychotic agents for example in the treatment of schizophrenia, schizo-affective disorders, schizophreniform diseases, psychotic depression, mania, acute mania, paranoid and delusional disorders. Furthermore, they may have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D₃ receptors, it could also be envisaged that the compounds could also have utility for the treatment of substance abuse where it has been suggested that D3 receptors are involved (e.g. see Levant, 1997, Pharmacol. Rev., 49, 231-252). Examples of such substance abuse include alcohol, cocaine, heroin and nicotine abuse. Other conditions which may be treated by the compounds include dyskinetic disorders such as Parkinson's disease, neuroleptic-induced parkinsonism and tardive dyskinesias; depression; anxiety; agitation; tension; social or emotional withdrawal in psychotic patients; cognitive impairment including memory disorders such as Alzheimer's disease; psychotic states associated with neurodegenerative disorders, e.g. Alzheimer's disease; eating disorders; obesity; sexual dysfunction; sleep disorders; emesis; movement disorders; obsessive-compulsive disorders; amnesia; aggression; autism; vertigo; dementia; circadian rhythm disorders; and gastric motility disorders e.g. IBS. Therefore, the invention provides a compound of formula (I) as hereinbefore described or a pharmaceutically acceptable salt or solvate thereof for use in therapy.

The invention also provides a compound of formula (I) or a pharmaceutically acceptable salt or solvate thereof for use in a condition which requires modulation of a dopamine receptor.

The invention also provides a compound of formula (I) as hereinbefore described or a pharmaceutically acceptable salt or solvate thereof for use in the treatment of psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides the use of a compound of formula (I) as hereinbefore described or a pharmaceutically acceptable salt or solvate thereof in the manufacture of a medicament for the treatment of a condition which requires modulation of a dopamine receptor.

The invention also provides the use of a compound of formula (I) as hereinbefore described or a pharmaceutically acceptable salt or solvate thereof in the manufacture of a medicament for the treatment of psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides a method of treating a condition which requires modulation of a dopamine receptor, which comprises administering to a mammal in need thereof an effective amount of a compound of formula (I) as hereinbefore described or a pharmaceutically acceptable salt or solvate thereof.

In a further aspect, the invention provides a method of treating psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

A preferred use for dopamine antagonists according to the present invention is in the treatment of psychotic disorders, Parkinsons disease, substance abuse, dyskinetic disorders, depression, bipolar disorder, anxiety and cognitive impairment.

“Treatment” includes prophylaxis, where this is appropriate for the relevant condition(s).

For use in medicine, the compounds of the present invention are usually administered as a standard pharmaceutical composition. The present invention therefore provides in a further aspect a pharmaceutical composition comprising a compound of formula (I) as hereinbefore described or a pharmaceutically (i.e. physiologically) acceptable salt thereof and a pharmaceutically (i.e. physiologically) acceptable carrier. The pharmaceutical composition can be for use in the treatment of any of the conditions described herein.

The compounds of formula (I) may be administered by any convenient method, for example by oral, parenteral (e.g. intravenous), buccal, sublingual, nasal, rectal or transdermal administration and the pharmaceutical compositions adapted accordingly.

The compounds of formula (I) as hereinbefore described and their pharmaceutically acceptable salts which are active when given orally can be formulated as liquids or solids, for example syrups, suspensions or emulsions, tablets, capsules and lozenges.

A liquid formulation will generally consist of a suspension or solution of the compound or pharmaceutically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochlorohydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

Each dosage unit for oral administration contains preferably from 1 to 250 mg (and for parenteral administration contains preferably from 0.1 to 25 mg) of a compound of the formula (I) or a pharmaceutically acceptable salt thereof calculated as the free base.

The pharmaceutically acceptable compounds of the invention will normally be administered in a daily dosage regimen (for an adult patient) of, for example, an oral dose of between 1 mg and 500 mg, preferably between 10 mg and 400 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a pharmaceutically acceptable salt thereof calculated as the free base, the compound being administered 1 to 4 times per day. Suitably the compounds will be administered for a period of continuous therapy, for example for a week or more.

Biological Test Methods

Binding Experiments on Cloned Dopamine (e.g. D₂ and D₃) Receptors

The ability of the compounds to bind selectively to human D₂/D₃ dopamine receptors can be demonstrated by measuring their binding to cloned receptors. The inhibition constants (Kᵢ) of test compounds for displacement of [¹²⁵I]-Iodosulpride binding to human D₂/D₃ receptors expressed in CHO cells were determined as follows. The cell lines were shown to be free from bacterial, fungal and mycoplasmal contaminants, and stocks of each were stored frozen in liquid nitrogen. Cultures were grown as monolayers or in suspension in standard cell culture media. Cells were recovered by scraping (from monolayers) or by centrifugation (from suspension cultures), and were washed two or three times by suspension in phosphate buffered saline followed by collection by centrifugation. Cell pellets were stored frozen at −80° C. Crude cell membranes were prepared by homogenisation followed by high-speed centrifugation, and characterisation of cloned receptors achieved by radioligand binding.

Preparation of CHO Cell Membranes

Cell pellets were gently thawed at room temperature, and resuspended in about 20 volumes of ice-cold Extraction buffer; 5 mM EDTA, 50 mM Trizma pre-set crystals (pH7.4@37° C.), 1 mM MgCl₂, 5 mM KCl and 120 mM NaCl. The suspension was homogenised using an Ultra-Turrax at fall speed for 15 seconds. The homogenate was centrifuged at 18,000 r.p.m for 15 min at 4° C. in a Sorvall RC5C centrifuge. Supernatant was discarded, and homogenate re-suspended in extraction buffer then centrifugation was repeated. The final pellet was resuspended in 50 mM Trizma pre-set crystals (pH 7.4 @ 37° C.) and stored in 1 ml aliquot tubes at −80° C. (D2=3.0E+08 cells, D3=7.0E+07 cells and D4=1.0E+08 cells). The protein content was determined using a BCA protocol and bovine serum albumin as a standard (Smith, P. K., et al., Measurement of protein using bicinchoninic acid. Anal. Biochem. 150, 76-85 (1985)).

Binding Experiments

Binding Experiments on D₃/D₂ Receptors

Crude D₂/D₃ cell membranes were incubated with 0.03 nM [¹²⁵I]-Iodosulpride (˜2000 Ci/mmol; Amersham, U. K., and the test compound in a buffer containing 50 mM Trizma pre-set crystals (pH 7.4 @ 37° C.), 120 mM NaCl, 5 mM KCl, 2 mM CaCl₂, 1 mM MgCl₂, 0.3% (w/v) bovine serum albumin. The total volume is 0.2 ml and incubated in a water bath at 37° C. for 40 minutes. Following incubation, samples were filtered onto GF/B Unifilters using a Canberra Packard Filtermate, and washed four times with ice-cold 50 mM Trizma pre-set crystals (pH 7.4 @ 37° C.). The radioactivity on the filters was measured using a Canberra Packard Topcount Scintillation counter. Non-specific binding was defined with 10 μM SKF-102161 (YM-09151). For competition curves, 10 serial log concentrations of competing cold drug were used (Dilution range: 10 μM-10 μM). Competition curves were analysed using Inflexion, an iterative curve fitting programme in Excel. Results were expressed as pKᵢ values where
pKᵢ=−log10[Ki].

The exemplified compounds have pKᵢ values within the range of 6.6-9.6 at the dopamine D₃ receptor.

The exemplified compounds have pKᵢ values within the range of 5.3-9.3 at the dopamine D₂ receptor.

Binding Experiments on Cloned 5-HT₆ Receptors

Compounds were tested following the procedures outlined in WO 98/27081. All of the exemplified compounds have pKᵢ values within the range of 7.0-8.8 at the serotonin 5-HT₆ receptor.

Binding Experiments on Cloned 5-HT₆ Receptors

Compounds were tested following the procedures outlined in WO 94/04533. All of the exemplified compounds have pKᵢ values within the range of 6.6-8.4 at the serotonin 5-HT_{6C} receptor.

Binding Experiments on Cloned 5-HT_{2A} Receptors

Compounds can be tested following the procedures outlined in British Journal of Pharmacology (1996) 117, 427-434. All of the exemplified compounds have pKi values within the range of 6.3-8.9 at the serotonin 5-HT_{2A} receptor.

The invention is further illustrated by the following non-limiting examples:

### DESCRIPTION 1

### 1-(7-Amino-1,2,4,5-tetrahydro-3-benzazepin-3-yl)-2,2,2-trifluoro-ethanone (D1)

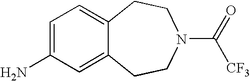

### 7-Nitro-1,2,4,5-tetrahydro-3H-3-benzazepine (D1a)

1,2,4,5-Tetrahydro-3H-benzazepine (1 g) (See P. Ruggli et al., Helv. Chim. Acta, 18, 1388, [1935]) was added slowly dropwise to stirred fuming nitric acid (25 ml) at −10° C. Stirring was continued at −10° C. for 1 hour and the reaction mixture was then poured onto ice, the precipitate collected by filtration and dried to give the title compound as the nitrate salt, 1.4 g. This salt was suspended in water, cooled to 5° C. and neutralised with 5M sodium hydroxide. The precipitate was collected by filtration, recrystallised from water and dried, affording the title compound D1a as a white solid (0.6 g).

### 1-(7-Nitro-1,2,4,5-tetrahydro-3-benzazepin-3-yl)-2,2,2-trifluoro-ethanone (D1b)

The 7-nitro benzazepine derivative (5 g) was dissolved in dichloromethane (80 ml) and to this was added diisopropylethylamine (5.4 ml) in dichloromethane (20 ml) at 0° C., followed by a solution of trifluoroacetic anhydride (4.3 ml) in dichloromethane (20 ml) at 0° C. The mixture was allowed to warm to room temperature and stirred overnight. Aqueous work up with water and dichloromethane gave the title compound D1b (7.0 g). MH⁺ 289

### 1-(7-Amino-1,2,4,5-tetrahydro-3-benzazepin-3-yl)-2,2,2-trifluoro-ethanone (D1)

The nitro derivative D1b was hydrogenated in accordance with the procedure described in D2c to give the title compound D1. M⁺ 259

### DESCRIPTION 2

### 7-Amino-1,2,3,4-tetrahydro-2-trifluoracetyl-isoquinoline (D2)

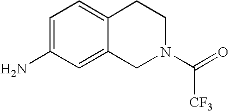

### N-2-(4-Nitrophenyl)ethyl-trifluoroacetamide (D2a)

A solution of trifluoroacetic anhydride (10.6 ml) in dichloromethane (100 ml) was added dropwise to a stirred solution of 2,6-lutidine (17.44 ml) and 4-nitrophenethylamine hydrochloride (15.2 g; 75 mmol) at 0° C. The mixture was stirred at 25° C. overnight under argon and then washed with dilute citric acid (2×), brine and dried over Na₂SO₄. The material in the organic phase gave the title compound D2a as a pale yellow solid (19.04 g).

### 7-Nitro-1,2,3,4-tetrahydro-2-trifluoracetyl-isoquinoline (D2b)

The nitro compound D2a (2.26 g; 9.15 mmol) and paraformaldehyde (0.45 g; 14.4 mmol) in acetic acid (10 ml) and conc. H₂SO₄ (15 ml) were stirred at 25° C. for 20 h according to the procedure of G. E. Stokker., Tet. Lett., 1996, 37, 5453. Work up afforded the title compound D2b as a white solid (2.17 g). ¹H NMR (CDCl₃) δ: 3.10 (2H, m), 3.92 (2H, m), 4.85+4.92 (2H, 2 xs), 7.38 (1H, t), 8.10 (2H, m). ^{m}/_{z} (EI): 274 (M⁺).

### 7-Amino-1,2,3,4-tetrahydro-2-trifluoracetyl-isoquinoline (D2)

The 7-nitro compound D2b (0.99 g, 3.6 mmol) in ethanol (50 ml) was hydrogenated over 10% palladium on carbon (450 mg) at atmospheric pressure for 4 h. The catalyst was removed by filtration through a pad of celite and evaporation gave the title compound D2 as a colourless solid (840 mg). ¹H NMR (CDCl₃) δ: 2.84 (2H, t), 3.23 (2H, bs), 3.82 (2H, m), 4.66 (2H, d), 6.47 (1H, m), 6.57 (1H, m), 6.96 (1H, m).

### DESCRIPTION 3

### 7-Amino-1,2,4,5-tetrahydro-3-benzazepine-3-carboxylic acid tert-butyl ester (D3)

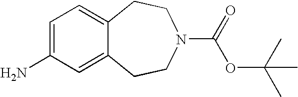

The title compound D3 was prepared using a similar methodology to that described in EP 284384. MH⁺ 263

### DESCRIPTION 4

### 7-Amino-2-(t-butyloxycarbonyl)-1,2,3,4-tetrahydroisoquinoline (D4)

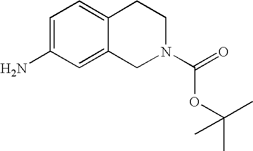

### 7-Nitro-1,2,3,4-tetrahydroisoquinoline (D4a)

The trifluoroacetamide D2b (17.22 g; 63 mmol) was hydrolysed at room temperature using a solution of potassium carbonate (46.6 g) in 10% aqueous methanol (660 ml). Work-up with dichloromethane gave the title compound D4a (11 g).

### 7-Amino-2-(t-butyloxycarbonyl)-1,2,3,4-tetrahydroisoquinoline (D4)

The title compound D4 was prepared from the compound D4a using di-t-butyl dicarbonate in 10% aqueous hydroxide in dioxan at 25° C. followed by catalytic hydrogenation according to the procedure described for D2c. MH⁺ 249.

### DESCRIPTION 5

### 7-Amino-8-methoxy-1,2,4,5-tetrahydro-3-benzazepine-3-carboxylic acid tert-butyl ester (D5)

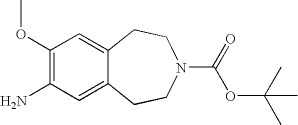

### 7-Methoxy-1,2,4,5-tetrahydro-3-benzazepine-3-carboxylic acid tert-butyl ester (D5a)

To a solution of 7-hydroxy-1,2,4,5-tetrahydro-3-benzazepine-3-carboxylic acid tert-butyl ester (5 g, 19 mmol) in dimethylformamide (50 ml) was added potassium carbonate (3.4 g, 25 mmol) and methyl iodide (3.25 ml, 60 mmol). The mixture was heated to 30° C. for 12 h. The solvent was evaporated and the residue partitioned between dichloromethane (100 ml) and water (100 ml). The organic layer was separated and evaporated to give the crude product D5a as a colourless oil (5.3 g, 100%).

### 7-Methoxy-8-nitro-1,2,4,5-tetrahydro-3-benzazepine-3-carboxylic acid tert-butyl ester (D5b)

To a mixture of 7-methoxy-1,2,4,5-tetrahydro-3-benzazepine-3-carboxylic acid tert-butyl ester (D5a) (5.3 g, 19 mmol) in glacial acetic acid (100 ml) and acetic anhydride (10 ml) at 0° C. was added a mixture of nitric acid (70% aqueous, 5 g, 55 mmol) dropwise in glacial acetic acid (100 ml) and acetic anhydride (10 ml) maintaining the temperature below 5° C. The mixture was stirred at room temperature for 2 h and then poured into ice/water (500 ml). The aqueous was extracted with dichloromethane (2×200 ml) and the combined organic portions were neutralised with saturated sodium bicarbonate solution. The dichloromethane layer was evaporated and the residue chromatographed on silica gel (eluent: hexane/dichloromethane (1:1) to dichloromethane) to give the product D5b as a colourless solid (1.5 g, 25%).

### 7-Amino-8-methoxy-1,2,4,5-tetrahydro-3-benzazepine-3-carboxylic acid tert-butyl ester (D5)

To a solution of 7-methoxy-8-nitro-1,2,4,5-tetrahydro-3-benzazepine-3-carboxylic acid tert-butyl ester D2b (1.5 g, 4.7 mmol) in ethanol (80 ml) was added palladium on charcoal (10%, 0.5 g). The mixture was stirred under an atmosphere of hydrogen for 2 h and then filtered. The solvent was evaporated to give the title compound D5 as a colourless solid (1.35 g, 100%).

Mass spectrum AP⁺: Found 193 ([M-Boc]⁺). C₁₆H₂₄N₂O₃ requires 292. ¹H NMR (CDCl₃) δ1.48 (9H, s), 2.76 (4H, m), 3.51 (4H, m), 3.65 (2H, s), 3.82 (3H, s), 6.50 (1H, m), 6.56 (1H, m).

### DESCRIPTION 6

### 5-Amino-1,3-dihydro-isoindole-2-carboxylic acid tert-butyl ester (D6)

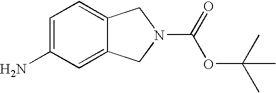

### 5-Nitroisoindoline nitrate (D6a)

Isoindoline (4 g, 33.1 mmol) was added to 95%. sulphuric acid, the reaction was treated carefully with fuming nitric acid (2.2 ml) at 0° C. and stirred for 1 h, then the mixture was poured onto ice and the resulting precipitate was collected by filtration and dried in vacuo to afford the title compound D6a (4.1 g, 46%); ¹H NMR (DMSO-d⁶) 8.35 (1H, s), 8.35 (1H, d, 8.4 Hz), 7.70 (1H, d, 8.4 Hz), 4.64 (4H,s).

### 5-Nitro-1,3-dihydro-isoindole-2-carboxylic acid tert-butyl ester (D6b)

The compound D6a (3.06 g, 13.47 mmol) in dichloromethane (50 ml) was treated with triethylamine (4.09 g, 40.42 mmol) followed by di-tertbutyl dicarbonate (3.08 g, 14.15 mmol) and stirred at room temperature for 3 days. The reaction was then diluted with dichloromethane and washed with 3N citric acid, sodium bicarbonate solution, water and brine. The organic phase was separated, dried over anhydrous sodium sulfate and evaporated in vacuo to afford the title compound D6b (3.5 g, 98%); ¹H NMR (CDCl₃) 8.19 (2H, m), 7.26 (1H, m), 4.75 (4H, m), 1.52 (9H, s).

### 5-Amino-1,3-dihydro-isoindole-2-carboxylic acid tert-butyl ester (D6)

The compound D6b (3.5 g, 13.25 mmol) was dissolved in ethanol (200 ml) and treated with 10 wt % Palladium on charcoal (1 g), and stirred under 1 atm of H₂ for 16 hours. The reaction was filtered and evaporated in vacuo to afford the title compound D6 (3.01 g, 96%);

MS (ES+), m/e 235 [MH]⁺.¹H NMR: δ CDCl₃ 1.52 (9H, s), 4.74 (2H, s), 4.77 (2H, s), 7.4 (1H, m), 8.2 (2H, m).

### DESCRIPTION 7

### 7-(4-Iodo-benzenesulfonylamino)-1,2,4,5-tetrahydro-3-benzazepine-3-carboxylic acid tert-butyl ester (D7)

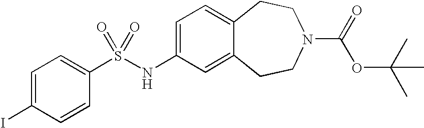

To a solution of D3 (4.7 g, 18 mmol) in pyridine (40 ml) at 0° C. was added dropwise a solution of 4-iodophenylsulfonyl chloride (6.1 g, 20 mmol) in dichloromethane (20 ml). The reaction mixture was then stirred at room temperature for 18 h, then poured onto brine. This mixture was extracted with ethyl acetate (3×), and the combined organic layers washed with citric acid solution, sodium bicarbonate solution then brine. The organic layer was dried over sodium sulfate then evaporated to afford the crude product. Chromatography on silica, eluting with 20-50% ethyl acetate/hexane afforded the title compound D7 (8 g). MH⁺ 529

### DESCRIPTION 8

### 4′-Chloro-biphenyl-4-sulfonyl chloride (D8)

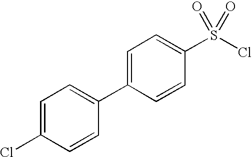

The title compound D8 was prepared from 4-chlorobiphenyl by chlorosulfonation with chlorosulfonic acid using the classical procedure (J. Med. Chem. 2000, 43, 156-166).

### DESCRIPTION 9

### 4′-Chloro-2-methyl-biphenyl-4-ylamine hydrochloride (D9)

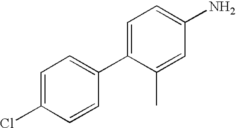

A mixture of 4-chlorophenyl boronic acid (6.32 g), 3-methyl-4-bromoaniline (5 g), toluene (135 ml), ethanol (40 ml) and potassium carbonate solution (40 ml) was degassed and then stirred under an atmosphere of argon. Tetrakis(triphenylphosphine)palladium(O) (0.62 g) was added and the mixture was stirred at reflux for 18 hours. The mixture was treated with water and ethyl acetate, then the organic layer was separated, washed with brine and evaporated. The residue was chromatographed on silica eluted with 10% ethyl acetate in hexane, and treated with hydrogen chloride in ether to give the title compound D9 as a white solid. ¹H NMR: δ DMSO-d⁶ 2.23 (3H, s), 7.2 (3H, m), 7.4 (2H, d), 7.5 (2H, d)

### DESCRIPTION 10

### 4′-Chloro-2-methyl-biphenyl4-sulfonyl chloride (D10)

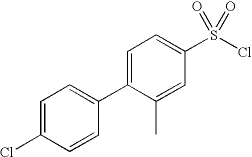

A stirred suspension of 4′-chloro-2-methyl-biphenyl-4-ylamine hydrochloride D9 (2.76 g) was cooled to −5° C. and treated with a solution of sodium nitrite (1.2 g) in water (10 ml). The resulting solution was stirred for 30 minutes, treated with urea (0.3 g) then added to a suspension of cuprous chloride (1 g) in acetic acid (30 ml) which had been saturated with sulfur dioxide stirred at 5° C. The solution was allowed to warm to room temperature over 1 hour, then heated to 40° C. for 30 minutes. Extraction with dichloromethane and chromatography on silica eluted with 5% ethyl acetate in hexane gave the title compound D10 as a white solid (1.65 g) ¹H NMR: δ CDCl₃ 2.37 (3H, s), 7.2 (2H, m), 7.4 (3H, m), 7.9 (2H, m).

### DESCRIPTION 11

### 7-Amino-8-ethoxy-1,2,4,5-tetrahydro-3-benzazepine-3-carboxylic acid tert-butyl ester (D11)

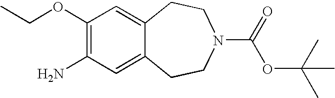

The title compound D11 was prepared in accordance with Description 5, but methyl iodide was replaced with ethyl iodide for the alkylation of the phenol ¹H NMR (CDCl₃) δ 6.55 (1H, s), 6.51 (1H, s), 4.03 (2H, q, J=7.0 Hz), 3.68 (2H, s), 3.51 (4H, m), 2.75 (4H, m), 1.48 (9H, s), 1.41 (3H, t, J=7.0 Hz).

### DESCRIPTION 12

### 7-Amino-8-isopropoxy-1,2,4,5-tetrahydro-3-benzazepine-3-carboxylic acid tert-butyl ester (D12)

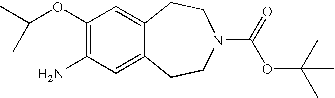

The title compound was prepared in accordance with Description 5, but methyl iodide was replaced with isopropyl iodide for the alkylation of the phenol. ¹H NMR (CDCl₃) δ 6.57 (1H, s), 6.50 (1H, s), 4.46 (1H, sept, J=6.1 Hz), 3.68 (2H, s), 3.51 (4H, m), 2.74 (4H, m), 1.48 (9H, s), 1.33 (6H, d, J=6.1 Hz).

### DESCRIPTION 13

### 7-Amino-8-bromo-1,2,4,5-tetrahydro-3-benzazepine-3-carboxylic acid tert-butyl ester (D13)

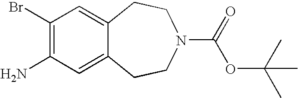

The aniline D3 (5 g, 19 mmol) was dissolved in dry acetonitrile (100 ml) and the solution was cooled to −15 ° C. A solution of N-bromosuccinimide (1.03 eq, 19.6 mmol, 3.48 g, in 70 ml of dry acetonitrile) was added dropwise at −15 ° C. to the solution containing the aniline, over 20 min. After the addition, the reaction mixture was left to warm up to room temperature for 10 min and then it was poured onto water/brine (150 ml+15 ml). The aqueous was extracted with EtOAc (100 ml, 50 ml), the organics were combined, dried over Na₂SO₄, filtered and the solvent was evaporated to afford the crude product. Chromatography on silica eluting with 5-30% EtOAc/n-hexane afforded the title compound D13 (1.3 g). M⁺-Boc)=241.

### DESCRIPTION 14

### 7-Amino-8-chloro-1,2,4,5-tetrahydro-3-benzazepine-3-carboxylic acid tert-butyl ester (D14)

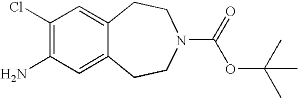

To a stirred solution of D3 (10 g, 38 mmol) in acetonitrile (300 ml) at 0° C. was added N-chlorosuccinimide (6.6 g, 49 mmol) portionwise over 10 minutes. The resulting solution was stirred overnight at room temperature, then water (500 ml) and EtOAc (500 ml) were added. The organic layer was separated, dried over magnesium sulfate and concentrated in vacuo to give a dark brown oil. This oil was purified by column chromatography using 20% diethyl ether/hexane as the eluant to give the title compound D14 as an orange glassy solid. (MH-Boc)⁺ 197.1, 199.1

### DESCRIPTION 15

### 7-Amino-8-ethyl-1,2,4,5-tetrahydro-benzo[d]azepine-3-carboxylic acid tert-butyl ester (D15)

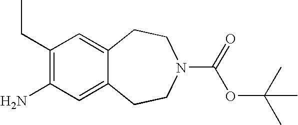

### 7-Hydroxy-1,2,4,5-tetrahydro-benzo[d]azepine-3-carboxylic acid tert-butyl ester (D15a)

The title compound was prepared according to the procedure described in WO 00/21951 i.e. 7-Methoxy-2,3,4,5-tetrahydro-1H-3-benzazepine (10 g) in 48% aqueous hydrobromic acid (350 ml) was allowed to stir at 100° C. for 4 h. The mixture was allowed to cool to 20° C. then evaporated to dryness, giving the crude hydroxy compound as a brown solid (14.5 g). This solid was dissolved in tetrahydrofuran (100 ml) and water (70 ml) and triethylamine (8 g) was added dropwise, followed by a solution of di-tert-butyl dicarbonate (14 g) in tetrahydrofuran (20 ml). The resulting mixture was allowed to stir at 20° C. for 16 h then partitioned between ethyl acetate (200 ml) and water (200 ml). The aqueous layer was extracted with ethyl acetate (100 ml). The combined organic extracts were washed with saturated aqueous sodium bicarbonate (100 ml), dried over anhydrous sodium sulfate and evaporated to dryness. The resulting oil was purified by chromatography over silica gel, eluting with 10-30% ethyl acetate in hexane, affording the title compound D15a as a white solid (8 g), MS (API⁺): Found 164 (MH⁺-Boc). C₁₅H₂₁NO₃ requires 263. ¹H NMR: δ CDCl₃ 1.48 (9H, s), 2.75-2.87 (4H, m), 3.40-3.60 (4H, m), 4.95 (1H, s), 6.50-6.62 (2H, m), 6.96 (1H, d).

### 7-Hydroxy-8-nitro-1,2,4,5-tetrahydro-benzo[d]azepine-3-carboxylic acid tert-butyl ester (D15b)

Nitration of D15a was carried out by adding 70% aqueous nitric acid (8 g) dissolved in glacial acetic acid (100 ml)/acetic anhydride (10 ml) to the phenol D15a (20 g) dissolved in AcOH (200 ml)/acetic anhydride (20 ml) at 0° C. Aqueous work-up followed by chromatography on silica gel using 0-20% EtOAc/n-hexane as eluant afforded the title compound D15b (11 g). ¹H NMR (CDCl₃) δ 7.85 (1H, s), 6.93 (1H, s), 3.56 (4H, m), 2.91 (4H, m), 1.48 (9H, m).

### 7-Nitro-8-trifluoromethanesulfonyloxy-1,2,4,5-tetrahydro-benzo[d]azepine-3-carboxylic acid tert-butyl ester (D15c)

D15b (8.4 g) was dissolved in acetone (300 ml) and cooled to 0° C. Trifluoromethanesulfonyl chloride (4.4 ml) was added and the resultant mixture stirred at room temperature for 2 h. Evaporation in vacuo followed by basic aqueous work-up afforded the title compound D15c (12 g). ¹H NMR (CDCl₃) δ 7.95 (1H, s), 7.19 (1H, s), 3.61 (4H, m), 3.02 (4H, m), 1.48 (9H, m).

### 7-Nitro-8-vinyl-1,2,4,5-tetrahydro-benzo[d]azepine-3-carboxylic acid tert-butyl ester (D15d)

A mixture of D15c (500 mg ), vinyl tri-n-butyltin (0.4 ml), lithium chloride (145 mg), palladium tetrakistriphenylphosphine (131 mg) and 2,6-di-tert-butylphenol (4 mg ) in 1,4-dioxan (4 ml) was heated at 160° C. for 0.5 h in a sealed tube in a Smith microwave reactor. Aqueous work-up followed by chromatography using 0-20% EtOAc/n-hexane as eluent gave the title compound D15d (260 mg).

### 7-Amino-8-ethyl-1,2,4,5-tetrahydro-benzo[d]azepine-3-carboxylic acid tert-butyl ester (D15)

Hydrogenation of D15d (260 mg) at 50 psi in ethanol (40 ml) over 10% palladium on charcoal (100 mg, paste) at room temperature afforded the title compound D15 (190 mg).

MH⁺291

### DESCRIPTION 16

### 7-Amino-8-methyl-1,2,4,5-tetrahydro-3-benzazepine-3-carboxylic acid tert-butyl ester (D16)

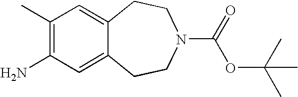

### 7-Methyl-8-nitro-1,2,4,5-tetrahydro[d]azepine-3-carboxylic acid tert-butyl ester (D16a)

A mixture of D15c (1.0 g), tetramethyltin (0.6 ml), lithium chloride (0.29 g), palladium tetrakistriphenylphosphine (0.13 g) and 2,6-di-tert-butylphenol (cat. ) in 1,4-dioxan (4 ml) was heated at 160° C. for 0.5 h in a sealed tube in a Smith microwave reactor. Aqueous work-up followed by chromatography using 0-20% EtOAc/n-hexane as eluent gave the title compound D16a (0.44 g).

### 7-Amino-8-methyl-1,2,4,5-tetrahydro-benzo[d]azepine-3-carboxylic acid tert-butyl ester (D16)

Hydrogenation of D16a (440 mg) at 50 psi in ethanol (100 ml) over 10% palladium on charcoal (200 mg, paste) at room temperature afforded the title compound D16 (330 mg).

(MH-Boc)⁺ 177.

### DESCRIPTION 17

### 7-Amino-8-ethylsulfanyl-1,2,4,5-tetrahydro-benzo[d]azepine-3-carboxylic acid tert-butyl ester (D17)

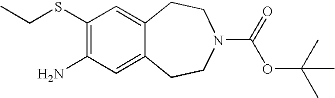

### 7-Nitro-8-ethylsulfanyl-1,2,4,5-tetrahydro-benzo[d]azepine-3-carboxylic acid tert-butyl ester (D17a)

A suspension of BINAP (106 mg), palladium(II) acetate (26 mg) and caesium carbonate (556 mg) in dioxan (5 ml) was sonicated for 30 min at room temperature. To the resulting red mixture was added D15c (0.5 g) and ethane thiol (0.2 ml) and the mixture was heated in a Smith microwave reactor for 30 mins at 160° C. The mixture was diluted with diethyl ether (30 ml) and water (30 ml) and the layers were separated. The aqueous portion was extracted with a further portion of diethyl ether (10 ml) and the combined organic extracts were washed with saturated sodium bicarbonate solution and then dried Na₂SO₄), filtered and evaporated. Chromatography using 0-10% EtOAc/n-hexane as eluent gave the title compound D17a (0.23 g).

### 7-Amino-8-ethylsulfanyl-1,2,4,5-tetrahydro-benzo[d]azepine-3-carboxylic acid tert-butyl ester (D17)

Hydrogenation of D17a (0.23 g) at 50 psi in ethanol (50 ml) over 10% palladium on charcoal (200 mg, paste) at room temperature afforded the title compound D17 (192 mg).

¹H NMR (CDCl₃) δ 7.12 (1H, s), 6.52 (1H, s), 4.23 (2H, m), 3.51 (4H, m), 2.72 (6H, m), 1.48 (9H, m), 1.22 (3H, t, J=7.4 Hz).

### DESCRIPTION 18

### 7-Amino-8-piperidin-1-yl-1,2,4,5-tetrahydro-benzo[d]azepine-3-carboxylic acid tert-butyl ester (D18)

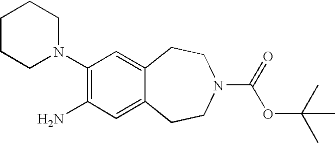

### 7-Nitro-8-piperidin-1-yl-1,2,4,5-tetrahydro-benzo[d]azepine-3-carboxylic acid tert-butyl ester (D18a)

A suspension of BINAP (106 mg), palladium(II) acetate (26 mg) and caesium carbonate (556 mg) in dioxan (5 ml) was sonicated for 30 min at room temperature. To the resulting red mixture was added D15c (0.5 g) and piperidine (0.2 ml) and the mixture was heated in a Smith microwave reactor for 30 mins at 160° C. The mixture was diluted with diethyl ether (30 ml) and water (30 ml) and the layers were separated. The aqueous portion was extracted with a further portion of diethyl ether (10 ml) and the combined organic extracts were washed with saturated sodium bicarbonate solution and then dried (Na₂SO₄), filtered and evaporated. Chromatography using 0-10% EtOAc/n-hexane as eluent gave the title compound D18a (0.28 g).

### 7-Amino-8-piperidin-1-yl-1,2,4,5-tetrahydro-benzo[d]azepine-3-carboxylic acid tert-butyl ester (D18)

Hydrogenation of D18a (278 mg) at 50 psi in ethanol (40 ml) over 10% palladium on charcoal (100 mg, paste) at room temperature afforded the title compound D18 (253 mg). MH⁺ 346

### DESCRIPTION 19

### 7-Amino-8-dimethylamino-1,2,4,5-tetrahydro-benzo[d]azepine-3-carboxylic acid tert-butyl ester (D19)

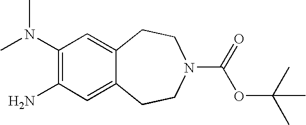

### 7-Nitro-8-dimethylamino-1,2,4,5-tetrahydro-benzo[d]azepine-3-carboxylic acid tert-butyl ester (D19a)

A suspension of BINAP (106 mg), palladium acetate (26 mg) and caesium carbonate (556 mg) in dioxan (5 ml) under argon was sonicated for 30 min at room temperature. To the resulting red suspension was added D15c (500 mg) and dimethylamine hydrochloride (150 mg). The mixture was then heated in a microwave reactor for 30 mins at 160° C., diluted with diethyl ether (30 ml) and washed with water (50 ml) and saturated sodium bicarbonate solution (30 ml) and then the layers separated. The organic portion was dried (Na₂SO₄), filtered and evaporated to give the title compound D19a as an oil (263 mg). MH⁺ 336

### 7-Amino-8-dimethylamino-1,2,4,5-tetrahydro-benzo[d]azepine-3-carboxylic acid tert-butyl ester (D19)

Hydrogenation of D19a at 50 psi in ethanol over 10% palladium on charcoal at room temperature afforded the title compound D19. MH⁺ 306

### DESCRIPTION 20

### 9-Chloro-3-methyl-2,3,4,5-tetrahydro-1H-benzo[d]azepin-7-ylamine (D20)

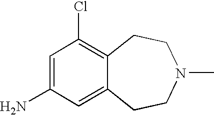

### 3-Acetyl-7-nitro-1,2,4,5-tetrahydro-3-benzazepine (D20a)

The title compound was prepared according to a similar procedure described in J. Heterocycl. Chem. 1971 8(5) 779.

### 3-Acetyl-7-nitro-9-iodo-1,2,4,5-tetrahydro-3-benzazepine (D20b)

D20a (22.4 g) in trifluoromethane sulphonic acid (150 ml) was treated with N-iodosuccinimide (40 g) portionwise over 5 days. Aqueous workup gave the crude title compound D20b (25 g). MH⁺ 361.

### 7-Nitro-9-iodo-1,2,4,5-tetrahydro-3-benzazepine (D20c)

Crude D20b (25 g) was heated to 120° C. in concentrated hydrochloric acid (1 liter) for 12 h. Basic aqueous workup followed by chromatography using 5% methanol/dichloromethane as eluent gave the title compound D20c (7 g). MH⁺ 319.

### 3-Methyl-7-nitro-9-iodo-1,2,4,5-tetrahydro-3-benzazepine (D20d)

D20c (7.3 g) was treated with formalin (37% aqueous, 20 ml) in dichloroethane (30 ml) for 0.5 h, followed by sodium triacetoxyborohydride (7 g). Chromatography using 1% methanol/dichloromethane as eluent and recrystallisation from dichloromethane/hexane gave the title compound D20d (1.9 g). MH⁺ 333.

### 3-Methyl-7-nitro-9-chloro-1,2,4,5-tetrahydro-3-benzazepine (D20e)

Reaction of D20d (0.8 g) with copper(I) chloride (1.68 g) in dimethylformamide (15 ml) at 120° C. for 2 h followed by chromatography using 1-3% methanol/dichloromethane as eluent gave the title compound D20e (0.3 g). MH⁺ 241.

### 9-Chloro-3-methyl-2,3,4,5-tetrahydro-1H-benzo[d]azepin-7-ylamine (D20)

Hydrogenation of D20e (0.3 g) at 1 atmosphere in ethanol over 10% rhodium on charcoal at room temperature afforded the title compound D20 (0.19 g). MH⁺ 211.

### DESCRIPTION 21

### 9-Bromo-3-methyl-2,3,4,5-tetrahydro-1H-benzo[d]azepin-7-ylamine (D21)

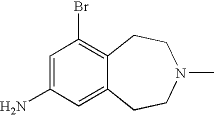

### 3-Methyl-7-nitro-9-iodo-1,2,4,5-tetrahydro-3-benzazepine (D21a)

The title compound was prepared according to the procedure described for D20d.

### 3-Methyl-7-nitro-9-bromo-1,2,4,5-tetrahydro-3-benzazepine (D21b)

Reaction of D21a (1 g) with copper(I) bromide (3 g) in dimethylformamide (10 ml) at reflux for 3 h followed by chromatography using 1-3% methanol/dichloromethane as eluent gave the title compound D21b (0.23 g). MH⁺ 286.

### 9-Bromo-3-methyl-2,3,4,5-tetrahydro-1H-benzo [d]azepin-7-ylamine (D21)

Reduction of the nitro group was achieved by treating D21b (0.23 g) in ethanol (6 ml), water (3 ml) and acetic acid (0.5 ml) with iron powder (180 mg) at reflux for 1 h. Basic aqueous workup and filtering gave the title compound D21 (0.19 g). MH⁺ 256.

### DESCRIPTION 22

### 7-(4-Iodo-benzenesulfonylamino)-8-methoxy-1,2,4,5-tetrahydro-benzo[d]azepine-3-carboxylic acid tert-butyl ester (D22)

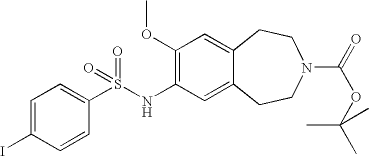

7-Amino-8-methoxy-1,2,4,5-tetrahydro-3-benzazepine-3-carboxylic acid tert-butyl ester (D5) (1.9 g, 6.5 mmol) was treated with pipsyl chloride (2.2 g, 7.2 mmol) in dichloromethane (20 ml) and pyridine (35 ml). The mixture was stirred for 13 h and the solvents evaporated. Chromatography on silica eluting with dichloromethane afforded the title compound D22 (2.8 g). M⁺-C(CH₃)₃+2H=503. ¹H NMR (CDCl₃) δ 7.76 (2H, d, J=8.6 Hz), 7.43 (2H, d, J=8.6 Hz), 6.81 (1H, s), 6.50 (1H, s), 3.58 (3H, s), 3.49 (4H, m), 2.80 (4H, m), 1.47 (9H, s).

### DESCRIPTION 23

### 7-[4-(4-Fluorobenzyl)benzenesulfonylamino]-1,2,4,5-tetrahydro-benzo[d]azepine-3-carboxylic acid tert-butyl ester (D23)

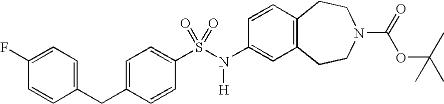

To a solution of the iodo compound D7 (0.129 g, 0.244 mmol, 1.0 eq) in anhydrous tetrahydrofuran (2 ml) under argon at room temperature was added dropwise 4-fluorobenzylzinc chloride (1.1 ml 0.5M in tetrahydrofuran, 0.537 mmol, 2.2 eq). The resultant solution was degassed by bubbling argon through the solution for 5 min then Pd(PPh₃)₄ was added and the solution heated at 50° C. for 4 h before allowing to cool to room temperature. Saturated aqueous NH₄Cl solution was added (10 ml) and the mixture extracted with EtOAc (2×10 ml). The organic layer was washed with brine (15 ml), dried over MgSO₄ and evaporated to dryness. Purification by chromatography over silica gel, eluting with 25% EtOAc-petrol afforded the title compound D23 as a pale yellow solid (0.120 g, 97%). MH⁺ 511. ¹H NMR δ CDCl₃ 1.47 (9H, s), 2.79 (4H, m), 3.48 (4H, m), 3.97 (2H, s), 6.44 (1H, s), 6.81 (2H, br.s), 6.82-7.25 (5H, m), 7.22 (2H, d), 7.67 (2H, d).

### DESCRIPTION 24

### 4-(4-Fluorobenzyl)-N-(2,3,4,5-tetrahydro-1H-benzo[d]azepin-7-yl)benzenesulfonamide hydrochloride (D24)

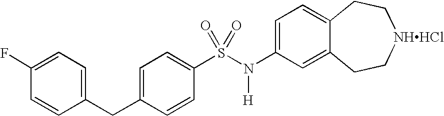

A solution of the Boc-protected amine D23 (0.104 g, 0.204 mmol, 1.0 eq) in 1,4 dioxan (3 ml) and 4M HCl in dioxan (2 ml, excess) was stirred at room temperature under argon for 6 h then evaporated to dryness, affording the desired compound D24 as a white solid (0.086 g, 96%). MH⁺ 411.

### EXAMPLE 1

### 4-(4-Chloro-phenyl)-N-(2,3,4,5-tetrahydro-1H-3-benzazepin-7-yl)-benzenesulfonamide (E1)

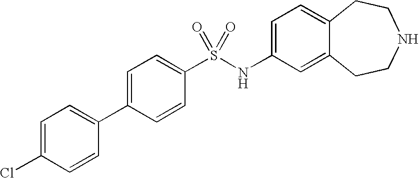

### 4-(4-Chloro-phenyl)-N-[3-(2,2,2-trifluoro-ethanoyl)-2,3,4,5-tetrahydro-1H-3-benzazepin-7-yl]-benzenesulfonamide (E1a)

A solution of 4′-chloro-biphenyl-4-sulfonyl chloride D8 (1.24 g, 4.3 mmol) in dichloromethane 910 ml) was added dropwise to a solution of D1 (1.0 g, 3.9 mmol) in pyridine (20 ml) at 0° C. The mixture was stirred at room temperature for 18 h, then poured onto brine and extracted with ethyl acetate (2×). The combined organic layer was washed with citric acid, sodium bicarbonate solution and brine, then dried and evaporated to afford the crude product. Chromatography on silica, eluting with 30% ethyl acetate/hexane afforded the product E1a (1.5 g). MH⁺ 509

### 4-(4-Chloro-phenyl)-N-(2,3,4,5-tetrahydro-1H-3-benzazepin-7-yl)-benzenesulfonamide (E1)

The compound E1a was dissolved in 2M ammonia in methanol (24 ml) and water (6 ml) added to the stirred solution. Stirring was continued for 18 h, then the solution evaporated to dryness. Application of the crude product to an SCX ion exchange cartridge, followed by elution with methanol followed by 1% ammonia in methanol afforded the title compound E1 (0.85 g). MH⁺ 413. ¹H NMR: δ CDCl₃ 2.8-2.9 (8H, m), 6.8 (2H, m), 6.96 (1H, d), 7.43 (2H, d), 7.50 (2H, d), 7.61 (2H, d), 7.81 (2H, d).

### EXAMPLE 2

### 4-(4-Chloro-phenyl)-N-(3-methyl-2,3,4,5-tetrahydro-1H-3-benzazepin-7-yl)-benzenesulfonamide (E2)

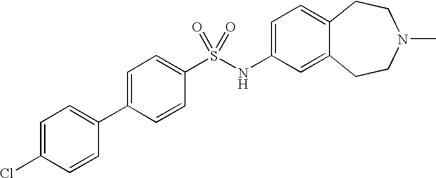

A solution of E1 (144 mg, 0.35 mmol) in dichloroethane (10 ml) was treated with formalin (0.3 ml) followed by sodium triacetoxyborohydride (250 mg). The mixture was stirred for 18 h, then added to sodium bicarbonate solution and extracted with dichloromethane. The combined organic extracts were washed with brine, dried and evaporated to afford the crude product. Chromatography on silica, eluting with 2% methanol in dichloromethane containing 0.5% aqueous ammonia, afforded the title compound E2 (140 mg). MH⁺ 425. ¹H NMR: δ CDCl₃ 2.35 (3H, s), 2.53 (4H, m), 2.86 (4H, m), 6.83 (2H, m), 6.96 (1H, d), 7.44 (2H, d), 7.51 (2H, d), 7.61 (2H, d), 7.81 (2H, d).

### EXAMPLE 3

### 4-(4-Chloro-phenyl)-N-methyl-N-(2,3,4,5-tetrahydro-1H-3-benzazepin-7-yl)-benzenesulfonamide (E3)

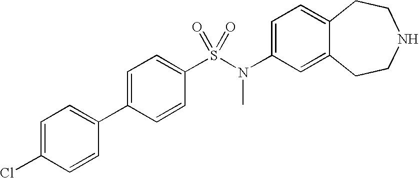

### 4-(4-Chloro-phenyl)-N-methyl-N-[3-(2,2,2-trifluoro-ethanoyl)-2,3,4,5-tetrahydro-1H-3-benzazepin-7-yl]-benzenesulfonamide (E3a)

The trifluoroacetamide E1a (500 mg, 1 mmol) was dissolved in dry tetrahydrofuran (15 ml) containing triphenylphosphine (330 mg) and dry methanol (200 mg). To this stirred solution was added di-isopropylazodicarboxylate (250 mg, 1.2 mmol) and the mixture stirred at room temperature for 18 h. The solvent was then evaporated and the residue chromatographed on silica using 20% ethyl acetate/hexane as eluant to afford the product E3a (640 mg). MH⁺ 523.

### 4(4-Chloro-phenyl)-N-methyl-N-(2,3,4,5-tetrahydro-1H-3-benzazepin-7-yl)-benzenesulfonamide (E3)

Deprotection of the compound D3a using a procedure similar to that for compound E1b afforded the title compound E3 (370 mg). MH⁺ 427. ¹H NMR: δ CDCl₃ 2.89 (8H, m), 3.18 (3H, s), 6.79 (1H, m), 6.91 (1H, s), 7.01 (1H, d), 7.46 (2H, d), 7.53 (2H, d), 7.65 (4H, s).

### EXAMPLE 4

### 4-(4-Chloro-phenyl)-N-methyl-N-(3-methyl-2,3,4,5-tetrahydro-1H-3-benzazepin-7-yl)-benzenesulfonamide (E4)

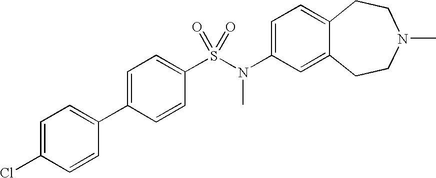

The title compound was prepared from E3 using a procedure similar to that for compound E2. MH⁺ 441. ¹H NMR: δ CDCl₃ 2.37 (3H, s), 2.57 (4H, s), 2.90 (4H, s), 3.18 (3H, s), 6.80 (1H, dd), 6.92 (1H, dd), 7.01 (1H, d), 7.45 (2H, d), 7.53 (2H, d), 7.63 (4H, s).

### EXAMPLE 5

### 4-(3,4-Dichloro-phenyl)-N-(2,3,4,5-tetrahydro-1H-3-benzazepin-7-yl)-benzenesulfonamide hydrochloride (E5)

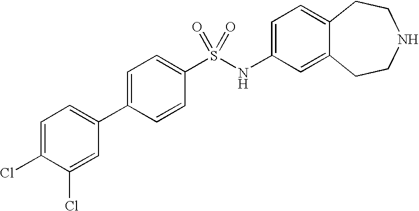

### 7-(3′,4′-Dichloro-biphenyl-4-sulfonylamino)-1,2,4,5-tetrahydro-3-benzazepine-3-carboxylic acid tert-butyl ester (E5a)

A solution of the iodo intermediate D7 (0.53 g, 1 mmol) was dissolved in a mixture of ethanol (3 ml), toluene (10 ml) and 2M aqueous potassium carbonate solution (3 ml) containing 3,4-dichlorobenzeneboronic acid (0.29 g, 1.5 mmol). This mixture was rigorously degassed and an argon atmosphere introduced. Tetrakis(triphenylphosphine)palladium (0.1 g) was added, and the mixture heated to 90° C. for 18 h. After cooling, the solution was poured onto brine and extracted with ethyl acetate (2×). The organic layer was washed with brine dried and evaporated to afford the crude product. Chromatography on silica, eluting with 10-25% ethyl acetate/hexane afforded the title compound E5a (0.57 g). MH⁺ 548.

### 4-(3,4-Dichloro-phenyl)-N-(2,3,4,5-tetrahydro-1H-3-benzazepin-7-yl)-benzenesulfonamide hydrochloride (E5)

The title compound was prepared from compound E5a by treatment with a solution of ethanolic hydrogen chloride, followed by the addition of ether to precipitate the product E5. MH⁺ 447. ¹H NMR: δ DMSO 2.98 (4H, s), 3.08 (4H, s), 6.95 (2H, m), 7.06 (1H, d), 7.74 (2H, m), 7.8-7.9 (4H, m), 8.01 (1H, dd).

### EXAMPLE 6

### 4-(4-Chloro-phenyl)-N-(8-methoxy-2,3,4,5-tetrahydro-1H-3-benzazepin-7-yl)-benzenesulfonamide hydrochloride (E6)

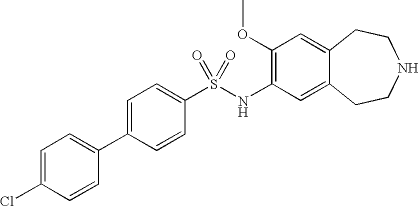

The title compound E6 was prepared from D5 and D8 using a procedure similar to that for compounds E1a and E5b. MH⁺ 443. ¹H NMR DMSO δ: 3.00 (4H, m), 3.11 (4H, m), 3.40 (3H, s), 6.79 (1H, s), 7.09 (1H, s), 7.56 (2H, d, J=8.5 Hz), 7.74 (2H, d, J=7.1 Hz), 7.77 (2H, d, J=7.1 Hz), 7.83 (2H, d, J=8.5 Hz), 9.14 (2H, s), 9.53 (1H, s)

### EXAMPLE 7

### 4-(4-Chloro-phenyl)-N-(8-methoxy-3-methyl-2,3,4,5-tetrahydro-1H-3-benzazepin-7-yl)-benzenesulfonamide hydrochloride (E7)

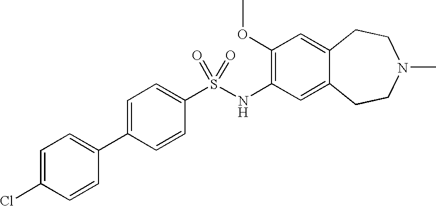

The title compound was prepared from E6 using a procedure similar to that for E2, and the product isolated as the hydrochloride salt. MH⁺ 457. ¹H NMR:δCDCl₃ 2.35 (3H, s), 2.50 (4H, m), 2.84 (4H, m), 3.57 (3H, s), 6.48 (1H, s), 6.9 (1H, b s), 7.31 (1H, s), 7.4-7.59 (6H, m), 7.80 (2H, m).

Examples 11-41 and 74-154 and 188-209 and 216-217 were prepared using analogous procedures to Examples 1-7 and 42-47 using the appropriate starting materials, with the products being isolated as either the free bases or hydrochloride salts. All ¹H NMR are consistent with the structures shown.

### EXAMPLE 8

### 4-(4-Chloro-phenyl)-N-(1,2,3,4-tetrahydro-isoquinolin-7-yl)-benzenesulfonamide (E8)

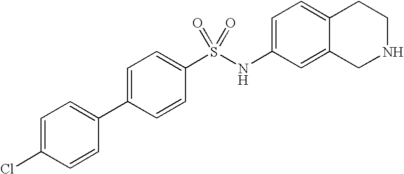

The title compound E8 was prepared from D4 and D8 using a procedure similar to that for compounds E1a and E5b. MH⁺ 399. ¹H NMR: δ DMSO-d⁶ 2.5 (2H,m), 2.8 (2H,m), 3.7 (2H, m), 6.77 (1H, ms), 6.9 (2H, m), 7.5 (2H, d), 7.7 (2H, d), 7.8(4H, m).

Examples 48-73 and 155-166 were prepared using analogous procedures to Examples 1-8 using the appropriate starting materials, with the products being isolated as either the free bases or hydrochloride salts. All ¹H NMR are consistent with the structures shown.

### EXAMPLE 9

### 4-(4-Chloro-phenyl)-N-(2,3-dihydro-1H-isoindol-5-yl)-benzenesulfonamide hydrochloride (E9)

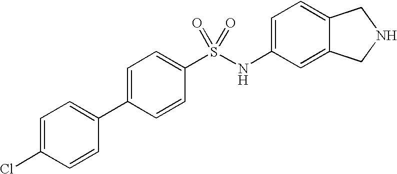

The title compound E9 was prepared from D6 and D8 using a procedure similar to that for compounds E1a and E5b. MH⁺ 385. ¹H NMR: δ DMSO-d⁶ 4.4 (4H, m), 7.11 (1H, d), 7.25 (2H, m), 7.55 (2H, d), 7.73 (2H, m), 7.86 (4H, s), 9.7 (2H, m), 10.55 (1H, m).

### EXAMPLE 10

### 4-(4-Chloro-phenyl)-N-(2-methyl-2,3-dihydro-1H-isoindol-5-yl)-benzenesulfonamide (E10)

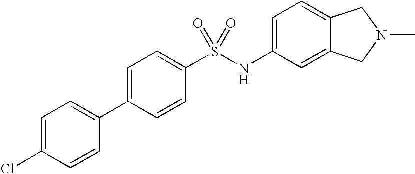

The title compound E10 was prepared from E9 using a procedure similar to that for compound E2. MH⁺ 399. ¹H NMR: δ DMSO-d⁶ 0.86 (3H, m), 1.2 (2H, m), 1.5 (2H, m), 2.41 (3H, s) 2.6 (2H, m), 3.68 (4H, s), 6.87 (IH, d), 6.93 (1H, s), 7.05 (2H, d), 7.32 (2H, d), 7.64 (2H, d).

Examples 167-174 were prepared using analogous procedures to Examples 9-10, and as described herein, using the appropriate starting materials, with the products being isolated as either the free bases or hydrochloride salts. All ¹H NMR are consistent with the structures shown.

### EXAMPLE 42

### 4-(4-Chloro-phenyl)-3-methyl-N-(2,3,4,5-tetrahydro-1H-3-benzazepin-7-yl)-benzenesulfonamide hydrochloride (E42)

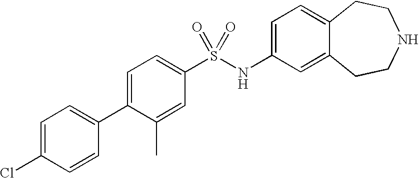

The title compound E42 was prepared from D3 and D9 using a procedure similar to that for compounds E1a and E5b. MH⁺ 427. ¹H NMR: δ DMSO-d⁶ 2.26 (3H,s), 3.0 (4H, m), 3.15 (4H, m), 6.95 (2H, m), 7.07 (1H, d), 7.4 (3H, m), 7.5 (2H, d), 7.63 (1H, d), 7.74 (1H, s), 9.1 (1H, br). 10.3 (1H, br)

### EXAMPLE 43

### 4-(4-Chloro-phenyl)-3-methyl-N-(3-methyl-2,3,4,5-tetrahydro-1H-3-benzazepin-7-yl)-benzenesulfonamide (E43)

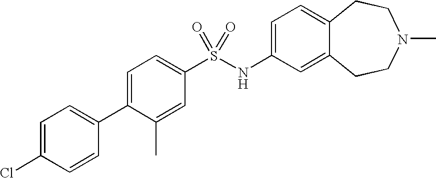

The title compound was prepared from E42 using a procedure similar to that for compound E2. MH⁺ 441. ¹H NMR: δ CDCl₃ 2.24 (3H,s), 2.34 (3H,s), 2.6 (4H, m), 2.8 (4H, m), 6.85 (2H, m), 7.0 (1H, d), 7.2 (3H, m), 7.4(2H, m), 7.6 (2H, m).

### EXAMPLE 44

### 4-(4-Chloro-phenyl)-3-methyl-N-(8-methoxy-2,3,4,5-tetrahydro-1H-3-benzazepin-7-yl)-benzenesulfonamide hydrochloride (E44)

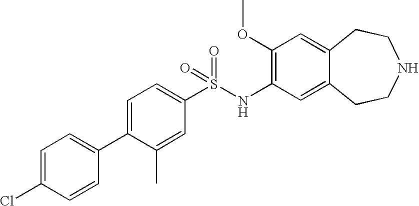

The title compound E44 was prepared from D5 and D10 using a procedure similar to for compounds E1a and E5b. MH⁺ 457. ¹H NMR: δ DMSO-d⁶ 2.51 (3H, s), 3.23 (8H, b s), 3.69 (3H, s), 6.57 (1H, s), 6.98 (1H, s), 7.20 (2H, m), 7.38 (3H, m), 7.60 (1H, d), 7.67 (1H, s).

### EXAMPLE 45

### 4-(4-Chloro-phenyl)-3-methyl-N-(8-methoxy-3-methyl-2,3,4,5-tetrahydro-1H-3-benzazepin-7-yl)-benzenesulfonamide (E45)

The title compound E44 was prepared from E46 using a procedure similar to that for compound E2. MH⁺ 471. ¹H NMR: δ CDCl₃ 2.23 (3H, s), 2.50 (3H, s), 2.74 (4H, s), 2.99 (4H, s), 3.64 (3H, s), 6.52 (1H, s), 7.17 (2H, d), 7.26 (1H, d), 7.31 (1H, s), 7.38 (2H, d), 7.41 (1H, m), 7.66 (1H, m).

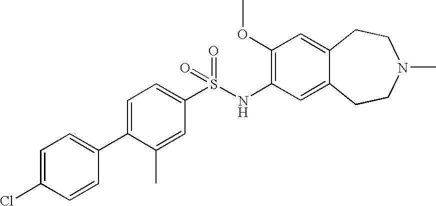

Examples 46-47 were prepared using analogous procedures to E44 and E45 using the appropriate starting materials, with the products being isolated as either the free bases or hydrochloride salts. All ¹H NMR are consistent with the structures shown.

### EXAMPLE 107

### 4-(5-Chloro-thiophen-2-yl)-N-(8-methoxy-3-methyl-2,3,4,5-tetrahydro-1H-benzo[d]azepin-7-yl)-benzenesulfonamide (E107)

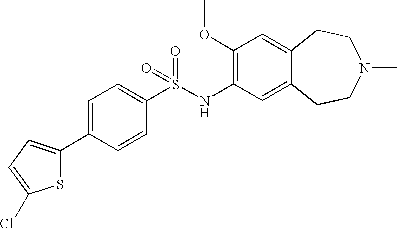

### 7-[4-(5-Chloro-thiophen-2-yl)-benzenesulfonylamino]-8-methoxy-1,2,4,5-tetrahydro-benzo[d]azepine-3-carboxylic acid tert-butyl ester (E107a)

7-(4-Iodo-benzenesulfonylamino)-8-methoxy-1,2,4,5-tetrahydro-benzo[d]azepine-3-carboxylic acid tert-butyl ester D22 (0.28 g, 0.5 mmol) was treated with 5-chloro-thiophene-2-boronic acid under standard Suzuki conditions (see D9) followed by aqueous workup and chromatography to give the title compound E107a (0.22 g). M⁺-C(CH₃)₃+H=493/495.

### 4-(5-Chloro-thiophen-2-yl)-N-(8-methoxy-2,3,4,5-tetrahydro-1H-benzo[d]azepin-7-yl)-benzenesulfonamide hydrochloride (E107b)

7-[4-(5-Chloro-thiophen-2-yl)-benzenesulfonylamino]-8-methoxy-1,2,4,5-tetrahydro-benzo[d]azepine-3-carboxylic acid tert-butyl ester E107a (0.22 g) was treated with 4M HCl in dioxan solution for 2 h. Diethyl ether was added and the precipitate filtered to give the title compound E107b as a colourless solid (0.19 g). M⁺ 447/449

### 4-(5-Chloro-thiophen-2-yl)-N-(8-methoxy-3-methyl-2,3,4,5-tetrahydro-1H-benzo[d]azepin-7-yl)-benzenesulfonamide (E107)

4-(5-Chloro-thiophen-2-yl)-N-(8-methoxy-2,3,4,5-tetrahydro-1H-benzo[d]azepin-7-yl)-benzenesulfonamide (E107b) (0.19 g) in dichloroethane (8 ml) was treated with triethylamine (0.9 ml) and formalin solution (37% aqueous, 0.3 ml) followed by sodium triacetoxyborohydride (250 mg). The mixture was shaken vigorously for 1 h and then diluted with dichloromethane (5 ml) and sodium bicarbonate solution (3 ml). The layers were separated and the organic portion evaporated. Chromatography on silica eluting with 10% methanol/ dichloromethane afforded the title compound E107 (57 mg). M⁺ 463/465

¹H NMR (CDCl₃) δ 7.71 (2H, d, J=8.5 Hz), 7.50 (2H, d, J=8.5 Hz), 7.29 (1H, s), 7.15 (1H, d, J=3.9 Hz), 6.92 (1H, d, J=3.9 Hz), 6.86 (1H, s), 6.48 (1H, s), 3.57 (3H, s), 2.88 (4H, m), 2.57 (4H, m), 2.39 (3H, s).

### EXAMPLE 216

### 4-(5-Chloro-thiophen-2-yl)-2-fluoro-N-(8-methoxy-3-methyl-2,3,4,5-tetrahydro-1H-benzo[d]azepin-7-yl)-benzenesulfonamide (E216)

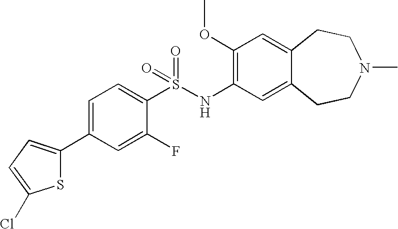

### 7-(4-Bromo-2-fluoro-benzenesulfonylamino)-8-methoxy-1,2,4,5-tetrahydro-benzo[d]azepine-3-carboxylic acid tert-butyl ester (E216a)

7-Amino-8-methoxy-1,2,4,5-tetrahydro-3-benzazepine-3-carboxylic acid tert-butyl ester D5 (391 mg) was treated with 2-fluoro-4-bromobenzenesulfonyl chloride (460 mg) in dichloromethane (15 ml) and pyridine (9 ml). The mixture was stirred for 3 h and the solvents evaporated. Chromatography on silica eluting with dichloromethane afforded the title compound E216a (740 mg). M−H 575

### 7-[2-Fluoro-4-(5-chloro-thiophen-2-yl)-benzenesulfonylamino]-8-methoxy-1,2,4,5-tetrahydro-benzo[d]azepine-3-carboxylic acid tert-butyl ester (E216b)

7-(4-Iodo-2-fluoro-benzenesulfonylamino)-8-methoxy-1,2,4,5 -tetrahydro-benzo[d]azepine-3-carboxylic acid tert-butyl ester E216a (320 mg) was treated with 5-chloro-thiophene-2-boronic acid (135 mg) under standard Suzuki conditions (see D9) followed by aqueous workup and chromatography to give the title compound E216b (140 mg). M−H 565

### 2-Fluoro-4-(5-Chloro-thiophen-2-yl)-N-(8-methoxy-2,3,4,5-tetrahydro-1H-benzo[d]azepin-7-yl)-benzenesulfonamide hydrochloride (E216c)

7-[2-Fluoro4-(5-chloro-thiophen-2-yl)-benzenesulfonylamino]-8-methoxy-1,2,4,5-tetrahydro-benzo[d]azepine-3-carboxylic acid tert-butyl ester (E216b) (140 mg) was treated with ethanolic HCl solution (6 ml) for 2 h. The solvent was evaporated to give the title compound E216c as a colourless solid (100 mg). M+H 445

### 4-(5-Chloro-thiophen-2-yl)-2-fluoro-N-(8-methoxy-3-methyl-2,3,4,5-tetrahydro-1H-benzo[d]azepin-7-yl)-benzenesulfonamide (E216)

2-Fluoro-4-(5-chloro-thiophen-2-yl)-N-(8-methoxy-2,3,4,5-tetrahydro-1H-benzo[d]azepin-7-yl)-benzenesulfonamide E216c (100 mg) in dichloroethane (8 ml) was treated with formalin solution (37% aqueous, 0.2 ml) followed by sodium triacetoxyborohydride (70 mg). The mixture was shaken vigorously for 1 h and then diluted with dichloromethane (5 ml) and sodium bicarbonate solution (5 ml). The layers were separated and the organic portion was evaporated. Chromatography on silica eluting with 10% methanol/dichloromethane afforded the title compound E216. M+H 459. ¹H NMR (DMSO-d⁶) (HCl salt) δ 10.78 (1H, s), 9.76 (1H, s), 7.79 (2H, d, J=11.5 Hz), 7.66 (1H, d, J=4 Hz), 7.59 (1H, t, J=8 Hz), 7.47 (1H, d, J=8 Hz), 7.26 (1H, d, J=4 Hz), 7.08 (1H, s), 6.81 (1H, s), 3.53 (2H, m), 3.42 (3H, s), 3.20 (2H, m), 2.92 (4H, m), 2.77 (3H, d, J=4.6 Hz).

### EXAMPLE 217

### 4′-Chloro-biphenyl-4-sulfonic acid (dimethylamino-methyl-2,3,4,5-tetrahydro-1H-benzo[d]azepin-7-yl)-amide (E217)

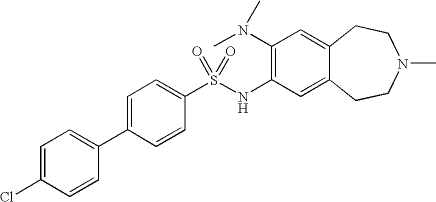

### 7-(4′-Chloro-biphenyl-4-sulfonylamino)-8-dimethylamino-1,2,4,5-tetrahydro-benzo[d]azepine-3-carboxylic acid dimethyl-ethyl ester (E217a)

7-Amino-8-dimethylamino-1,2,4,5-tetrahydro-3-benzazepine-3-carboxylic acid tert-butyl ester (D19) (120 mg) was treated with 4′-chlorobiphenyl4-sulfonyl chloride (136 mg) in dichloromethane (5 ml) and pyridine (0.05 ml). Mixture stirred for 3 h and the solvents evaporated. Chromatography on silica eluting with 20% ethyl acetate/hexane afforded the title compound E217a (175 mg). M+H 556/558

### 4′-Chloro-biphenyl-4-sulfonic acid (8-dimethylamino-2,3,4,5-tetrahydro-1H-benzo[d]azepin-7-yl)-amide hydrochloride (E217b)

7-(4′-Chloro-biphenyl-4-sulfonylamino)-dimethylamino-1,2,4,5-tetrahydro-benzo[d]azepine-3-carboxylic acid dimethyl-ethyl ester (E217a) (175 mg) was treated with ethanolic HCl solution (4 ml) for 2 h. The solvent was evaporated to give the title compound E217b as a colourless solid (120 mg). M+H 456/458

### 4′-Chloro-biphenyl-4-sulfonic acid (dimethylamino-methyl-2,3,4,5-tetrahydro-1H-benzo[d]azepin-7-yl)-amide (E217)

4′-Chloro-biphenyl-4-sulfonic acid (8-dimethylamino-2,3,4,5-tetrahydro-1H-benzo[d]azepin-7-yl)-amide hydrochloride (E217b) (75 mg) in dichloroethane (3 ml) was treated with formalin solution (37% aqueous, 1 ml) followed by sodium triacetoxyborohydride (48 mg). The mixture was shaken vigorously for 1 h and then diluted with dichloromethane (10 ml) and sodium bicarbonate solution (10 ml). The layers were separated and the organic portion was evaporated. Chromatography on silica eluting with 10% methanol/dichloromethane afforded the title compound E217 (65 mg). M+H 470/472. ¹H NMR (CDCl₃) δ 8.05 (1H, br s), 7.90 (2H, d, J=6.7 Hz), 7.60 (2H, d, J=6.7 Hz), 7.47 (2H, d, J=6.4 Hz), 7.42 (2H, d, J=6.4 Hz), 7.35 (1H, s), 6.83 (1H, s), 2.87 (2H, m), 2.81 (2H, m), 2.53 (4H, m), 2.40 (6H, s), 2.35 (3H, s).

### EXAMPLE 210

### 4-(4-Fluorobenzyl)-N-(3-methyl-2,3,4,5-tetrahydro-1H-benzo[d]azepin-7-yl)benzenesulfonamide hydrochloride (E210)

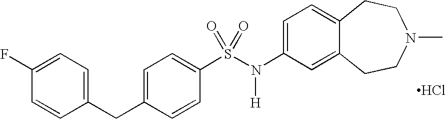

To a suspension of salt D24 (0.083 g, 0.186 mmol, 1.0 eq) in 1,2-dichloroethane (3.5 ml) at room temperature was added triethylamine (26 μl, 0.186 mmol, 1.0 eq) followed by 37% aqueous formaldehyde solution (0.6 ml, excess). After vigorous stirring for 5 min. sodium triacetoxyborohydride (0.090 g, excess) was added portionwise. After 2 h saturated aqueous sodium bicarbonate solution (10 ml) and dichloromethane (10 ml) were added and the layers separated. The organic layer was evaporated to dryness, affording the free base as a pale yellow solid (0.077 g, 97%). The solid was dissolved in methanol, 1M HCl added (1.05 eq) and the mixture concentrated to dryness, giving the title compound E210 as an off-white solid. MH⁺ 425. ¹H NMR δ DMSO-d⁶ 2.43 (3H, s), 2.82 (4H, m), 3.51 (4H, m), 4.04 (2H, s), 6.93-7.35 (7H, m), 7.39 (2H, d), 7.73 (2H, d), 10.28 (1H, s), 10.75 (1H, s).

Examples 175-187 were prepared using analogous procedures to Example 188 using the appropriate starting materials and Examples 211-215 using analogous procedures to Descriptions 23-24 and Example 210, with the products being isolated as either free bases or hydrochloride salts. All ¹H NMR are consistent with the structures shown.

All of the compounds listed below in Table 1 relate to compounds of the formula (IJ):

TABLE 1

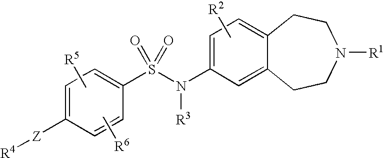

| Ex- | | | | | | | | |
|---|---|---|---|---|---|---|---|---|
| ample | R¹ | R² | R³ | R⁴ | R⁵ | R⁶ | Z | MH⁺ |
| 1 | H | H | H | 4-ClPh | H | H | bond | 413 |
| 2 | Me | H | H | 4-ClPh | H | H | bond | 427 |
| 3 | H | H | Me | 4-ClPh | H | H | bond | 427 |
| 4 | Me | H | Me | 4-ClPh | H | H | bond | 441 |
| 5 | H | H | H | 3,4-diClPh | H | H | bond | 447 |
| 6 | H | 8-MeO | H | 4-ClPh | H | H | bond | 443 |
| 7 | Me | 8-MeO | H | 4-ClPh | H | H | bond | 457 |
| 11 | H | 8-Br | H | 4-ClPh | H | H | bond | 493 |
| 12 | Me | H | H | 2-ClPh | H | H | bond | 427 |
| 13 | H | H | H | 3-ClPh | H | H | bond | 413 |
| 14 | Me | H | H | 3-ClPh | H | H | bond | 427 |
| 15 | Me | H | H | 3,4-diClPh | H | H | bond | 461 |
| 16 | Me | H | H | 2,4-diClPh | H | H | bond | 461 |
| 17 | H | H | H | 4-BrPh | H | H | bond | 458 |
| 18 | Me | H | H | 4-BrPh | H | H | bond | 472 |
| 19 | Me | H | H | 4-MePh | H | H | bond | 407 |
| 20 | H | H | H | 3-MePh | H | H | bond | 393 |
| 21 | Me | H | H | 3-MePh | H | H | bond | 407 |
| 22 | H | H | H | 2-MePh | H | H | bond | 393 |
| 23 | Me | H | H | 2-MePh | H | H | bond | 407 |
| 24 | Me | H | H | 4-CF₃Ph | H | H | bond | 461 |
| 25 | Me | H | H | 4-OCF₃Ph | H | H | bond | 477 |
| 26 | Me | H | H | 4-MeSPh | H | H | bond | 439 |
| 27 | Me | H | H | 4-t-BuPh | H | H | bond | 449 |
| 28 | H | H | H | 4-CNPh | H | H | bond | 405 |
| 29 | Me | H | H | 4-CNPh | H | H | bond | 419 |
| 30 | Me | H | H | 4-MeOPh | H | H | bond | 423 |
| 31 | Me | H | H | 4-FPh | H | H | bond | 411 |
| 32 | Me | H | H | 2-thienyl | H | H | bond | 399 |
| 33 | Me | H | H | 5-Cl-2-thienyl | H | H | bond | 434 |
| 34 | H | H | H | 3-thienyl | H | H | bond | 385 |
| 35 | Me | H | H | 3-thienyl | H | H | bond | 399 |
| 36 | Me | H | H | 2-naphthyl | H | H | bond | 443 |
| 37 | H | H | H | 2-benzofuranyl | H | H | bond | 419 |
| 38 | H | H | H | 4-pyridyl | H | H | bond | 379 |
| 39 | Et | H | H | 4-ClPh | H | H | bond | 441 |
| 40 | n-Pr | H | H | 4-ClPh | H | H | bond | 455 |
| 41 | i-Pr | H | H | 4-ClPh | H | H | bond | 455 |
| 42 | H | H | H | 4-ClPh | 3-Me | H | bond | 427 |
| 43 | Me | H | H | 4-ClPh | 3-Me | H | bond | 441 |
| 44 | H | 8-OMe | H | 4-ClPh | 3-Me | H | bond | 457 |
| 45 | Me | 8-OMe | H | 4-ClPh | 3-Me | H | bond | 471 |
| 46 | H | 8-Br | H | 4-ClPh | 3-Me | H | bond | 506 |
| 47 | Me | 8-Br | H | 4-ClPh | 3-Me | H | bond | 520 |
| 74 | Me | H | H | 4-NO₂Ph | H | H | bond | 438 |
| 75 | H | H | H | 3-furanyl | H | H | bond | 369 |
| 76 | Me | H | H | 3-furanyl | H | H | bond | 383 |
| 77 | Me | H | H | 4-ClPh | H | H | O | 443 |
| 78 | H | 8-MeO | H | Ph | H | H | bond | 409 |
| 79 | Me | 8-MeO | H | Ph | H | H | bond | 423 |
| 80 | H | 8-MeO | H | 3-ClPh | H | H | bond | 443 |
| 81 | Me | 8-MeO | H | 3-ClPh | H | H | bond | 457 |
| 82 | H | 8-MeO | H | 3,4-diClPh | H | H | bond | 478 |
| 83 | Me | 8-MeO | H | 3,4-diClPh | H | H | bond | 492 |
| 84 | H | 8-MeO | H | 2,4-dIClPh | H | H | bond | 478 |
| 85 | Me | 8-MeO | H | 2,4-diClPh | H | H | bond | 492 |
| 86 | H | 8-MeO | H | 2-Me-4-ClPh | H | H | bond | 457 |
| 87 | Me | 8-MeO | H | 2-Me-4-ClPh | H | H | bond | 471 |
| 88 | H | 8-MeO | H | 4-FPh | H | H | bond | 427 |
| 89 | Me | 8-MeO | H | 4-FPh | H | H | bond | 441 |
| 90 | H | 8-MeO | H | 4-CF₃Ph | H | H | bond | 477 |
| 91 | Me | 8-MeO | H | 4-CF₃Ph | H | H | bond | 491 |
| 92 | H | 8-MeO | H | 4-OCF₃Ph | H | H | bond | 493 |
| 93 | Me | 8-MeO | H | 4-OCF₃Ph | H | H | bond | 507 |
| 94 | H | 8-MeO | H | 4-MeOPh | H | H | bond | 439 |
| 95 | Me | 8-MeO | H | 4-MeOPh | H | H | bond | 453 |
| 96 | H | 8-MeO | H | 4-CNPh | H | H | bond | 434 |
| 97 | Me | 8-MeO | H | 4-CNPh | H | H | bond | 448 |
| 98 | H | 8-MeO | H | 4-(NMe₂)Ph | H | H | bond | 452 |
| 99 | Me | 8-MeO | H | 4-(NMe₂)Ph | H | H | bond | 466 |
| 100 | H | 8-MeO | H | Ph | H | H | O | 425 |
| 101 | Me | 8-MeO | H | Ph | H | H | O | 439 |
| 102 | H | 8-MeO | H | 4-ClPh | H | H | O | 459 |
| 103 | Me | 8-MeO | H | 4-ClPh | H | H | O | 473 |
| 104 | H | 8-MeO | H | 2-thienyl | H | H | bond | 415 |
| 105 | Me | 8-MeO | H | 2-thienyl | H | H | bond | 429 |
| 106 | H | 8-Me0 | H | 5-Cl-2-thienyl | H | H | bond | 449 |
| 107 | Me | 8-MeO | H | 5-Cl-2-thienyl | H | H | bond | 463 |
| 108 | H | 8-MeO | H | 3-thienyl | H | H | bond | 415 |
| 109 | Me | 8-MeO | H | 3-thienyl | H | H | bond | 429 |
| 110 | H | 8-Me0 | H | 3-furanyl | H | H | bond | 399 |
| 111 | Me | 8-MeO | H | 3-furanyl | H | H | bond | 413 |
| 112 | H | 8-MeO | H | 4-pyridyl | H | H | bond | 410 |
| 113 | Me | 8-MeO | H | 4-pyridyl | H | H | bond | 424 |
| 114 | H | H | H | 4-ClPh | 3-F | H | bond | 431 |
| 115 | Me | H | H | 4-ClPh | 3-F | H | bond | 445 |
| 116 | H | H | H | 4-ClPh | 3-Cl | H | bond | 448 |
| 117 | H | 8-EtO | H | 4-ClPh | H | H | bond | 457 |
| 118 | Me | 8-EtO | H | 4-ClPh | H | H | bond | 471 |
| 119 | H | 8-i-PrO | H | 4-ClPh | H | H | bond | 471 |
| 120 | Me | 8-i-PrO | H | 4-ClPh | H | H | bond | 485 |
| 121 | H | 8-EtO | H | 4-ClPh | 3-Me | H | bond | 472 |
| 122 | Me | 8-EtO | H | 4-ClPh | 3-Me | H | bond | 486 |
| 123 | H | 8-i-PrO | H | 4-ClPh | 3-Me | H | bond | 486 |
| 124 | Me | 8-i-PrO | H | 4-ClPh | 3-Me | H | bond | 500 |
| 125 | H | 8-i-PrO | H | 2-thienyl | H | H | bond | 443 |
| 126 | H | 8-i-PrO | H | 3-thienyl | H | H | bond | 443 |
| 127 | H | 8-i-PrO | H | 3-furanyl | H | H | bond | 427 |
| 128 | H | 8-i-PrO | H | 4-FPh | H | H | bond | 455 |
| 129 | H | 8-i-PrO | H | 4-MeOPh | H | H | bond | 467 |
| 130 | H | 8-i-PrO | H | 4-CF₃OPh | H | H | bond | 521 |
| 131 | H | 8-i-PrO | H | 2-Me-4-ClPh | H | H | bond | 486 |
| 132 | H | 8-i-PrO | H | 3-Me-4-ClPh | H | H | bond | 486 |
| 133 | Me | 8-i-PrO | H | 2-thienyl | H | H | bond | 457 |
| 134 | Me | 8-i-PrO | H | 3-thienyl | H | H | bond | 457 |
| 135 | Me | 8-i-PrO | H | 3-furanyl | H | H | bond | 441 |
| 136 | Me | 8-i-PrO | H | 4-FPh | H | H | bond | 469 |
| 137 | Me | 8-i-PrO | H | 4-MeOPh | H | H | bond | 481 |
| 138 | Me | 8-i-PrO | H | 4-CF₃OPh | H | H | bond | 535 |
| 139 | Me | 8-i-PrO | H | 2-Me-4-ClPh | H | H | bond | 500 |
| 140 | Me | 8-i-PrO | H | 3-Me-4-ClPh | H | H | bond | 500 |
| 141 | Me | 8-MeO | H | 4-ClPh | 2-F | H | bond | 475 |
| 142 | Me | 8-Br | H | 4-ClPh | 2-F | H | bond | 524 |
| 143 | Me | 8-MeO | H | 4-ClPh | 3-F | H | bond | 475 |
| 144 | Me | 8-MeO | H | 4-ClPh | 3-CF₃ | H | bond | 525 |
| 145 | H | H | i-Pr | 4-ClPh | H | H | bond | 455 |
| 146 | Me | H | i-Pr | 4-ClPh | H | H | bond | 469 |
| 147 | H | H | Me | 3-thienyl | H | H | bond | 399 |
| 148 | Me | H | Me | 3-thienyl | H | H | bond | 413 |
| 149 | H | H | Me | 4-CNPh | H | H | bond | 418 |
| 150 | Me | H | Me | 4-CNPh | H | H | bond | 432 |
| 151 | H | 8-MeO | i-Pr | 4-ClPh | H | H | bond | 485 |
| 152 | Me | 8-MeO | i-Pr | 4-ClPh | H | H | bond | 499 |
| 153 | H | 8-MeO | Me | 4-ClPh | H | H | bond | 457 |
| 154 | Me | 8-MeO | Me | 4-ClPh | H | H | bond | 471429 |
| 175 | Me | 8-MeO | H | 5-Me-2-thienyl | H | H | bond | 443 |
| 176 | Me | 8-Br | H | 5-Me-2-thienyl | H | H | bond | 492 |
| 177 | Me | 8-Br | H | 3,5-dimethylisoxazol-4-yl | H | H | bond | 491 |
| 178 | Me | 8-Br | ⁱPr | 3,5-dimethylisoxazol-4-yl | H | H | bond | 533 |
| 179 | Me | 8-Cl | H | 3,5-dimethylisoxazol-4-yl | H | H | bond | 446 |
| 180 | Me | 8-Cl | ⁱPr | 3,5-dimethylisoxazol-4-yl | H | H | bond | 489 |
| 181 | Me | 8-H | H | 5-Me-2-furyl | H | H | bond | 397 |
| 182 | Me | 8-Br | H | 5-Me-2-furyl | H | H | bond | 476 |
| 183 | Me | 8-Cl | H | 5-Me-2-furyl | H | H | bond | 431 |
| 184 | Me | 8-MeO | H | 5-Me-2-furyl | H | H | bond | 427 |
| 185 | Me | 8-MeO | H | 4-Me-2-thienyl | H | H | bond | 443 |
| 186 | Me | 8-H | H | N-Boc-2-pyrrolyl | H | H | bond | 412 |
| 187 | Me | 8-MeO | H | N-Boc-2-pyrrolyl | H | H | bond | 512 |
| 188 | H | 8-Et | H | 4-FPh | H | H | bond | 425 |
| 189 | Me | 8-Et | H | 4-ClPh | H | H | bond | 456 |
| 190 | Me | 8-Et | H | 4-FPh | H | H | bond | 439 |
| 191 | Me | H | H | 3,4-FPh | H | H | bond | 429 |
| 192 | Me | H | H | 2-FPh | H | H | bond | 411 |
| 193 | Me | 8-Et | iPr | 2-FPh | H | H | bond | 481 |
| 194 | Me | 8-SEt | H | 4-ClPh | H | H | bond | 488 |
| 195 | Me | 8-Me | H | 4-FPh | H | H | bond | 425 |
| 196 | Me | 8-Br | iPr | 2,4-FPh | H | H | bond | 550 |
| 197 | Me | 8-Br | iPr | 3,5-FPh | H | H | bond | 550 |
| 198 | Me | 8-Me₂N | H | 4-FPh | H | H | bond | 454 |
| 199 | Me | 8-Me | H | 4-ClPh | H | H | bond | 441 |
| 200 | Me | 8-Me | iPr | 4-ClPh | H | H | bond | 467 |
| 201 | Me | 8-Cl | H | 4-FPh | H | H | bond | 445 |
| 202 | Me | 8-EtS | H | 4-FPh | H | H | bond | 471 |
| 203 | Me | 8-piperidyl | H | 4-FPh | H | H | bond | 494 |
| 204 | Me | 9-Cl | H | 4-FPh | H | H | bond | 445 |
| 205 | Me | 9-Br | H | 4-FPh | H | H | bond | 490 |
| 206 | Et | 8-OMe | H | 2-thienyl-5Cl | H | H | bond | 478 |
| 207 | iPr | 8-OMe | H | 2-thienyl-5Cl | H | H | bond | 492 |
| 208 | iBu | 8-OMe | H | 2-thienyl-5Cl | H | H | bond | 506 |
| 209 | Bn | 8-OMe | H | 2-thienyl-5Cl | H | H | bond | 540 |
| 210 | Me | 8-H | H | 4-FPh | H | H | CH₂ | 425 |
| 211 | Me | 8-H | H | 3-FPh | H | H | CH₂ | 425 |
| 212 | Me | 8-MeO | H | 4-FPh | H | H | CH₂ | 455 |
| 213 | Me | 8-MeO | H | 3-FPh | H | H | CH₂ | 455 |
| 214 | Me | 8-Br | H | 4-FPh | H | H | CH₂ | 504 |
| 215 | Me | 8-Br | H | 3-FPh | H | H | CH₂ | 504 |
| 216 | Me | 8-MeO | H | 2-thienyl-5Cl | 2-F | H | bond | 481 |
| 217 | Me | 8-NMe₂ | H | 4-ClPh | H | H | bond | 470 |

All of the compounds listed below in Table 2 relate to compounds of the formula (IF):

TABLE 2

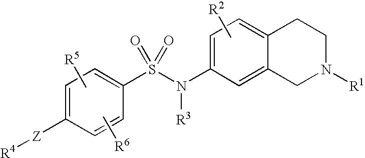

| Example | R¹ | R² | R³ | R⁴ | R⁵ | R⁶ | Z | MH⁺ |
|---|---|---|---|---|---|---|---|---|
| 8 | H | H | H | 4-ClPh | H | H | bond | 399 |
| 48 | Me | H | H | 4-ClPh | H | H | bond | 413 |
| 49 | Me | H | H | 2-ClPh | H | H | bond | 413 |
| 50 | H | H | H | 3-ClPh | H | H | bond | 399 |
| 51 | Me | H | H | 3-ClPh | H | H | bond | 413 |
| 52 | Me | H | H | 3,4-diClPh | H | H | bond | 447 |
| 53 | Me | H | H | 2,4-dIClPh | H | H | bond | 447 |
| 54 | H | H | H | 4-BrPh | H | H | bond | 444 |
| 55 | Me | H | H | 4-BrPh | H | H | bond | 458 |
| 56 | Me | H | H | 4-FPh | H | H | bond | 397 |
| 57 | H | H | H | 3-MePh | H | H | bond | 379 |
| 58 | Me | H | H | 3-MePh | H | H | bond | 393 |
| 59 | H | H | H | 4-CF₃Ph | H | H | bond | 433 |
| 60 | H | H | H | 4-OCF₃Ph | H | H | bond | 449 |
| 61 | Me | H | H | 4-OCF₃Ph | H | H | bond | 463 |
| 62 | H | H | H | 4-t-BuPh | H | H | bond | 421 |
| 63 | Me | H | H | 4-t-BuPh | H | H | bond | 435 |
| 64 | H | H | H | 5-Cl-2-thienyl | H | H | bond | 405 |
| 65 | Me | H | H | 5-Cl-2-thienyl | H | H | bond | 419 |
| 66 | H | H | H | 2-naphthyl | H | H | bond | 415 |
| 67 | Me | H | H | 2-naphthyl | H | H | bond | 429 |
| 68 | H | H | Me | 4-ClPh | H | H | bond | 413 |
| 69 | Me | H | Me | 4-ClPh | H | H | bond | 427 |
| 70 | H | H | H | 4-ClPh | 3-Me | H | bond | 413 |
| 71 | Me | H | H | 4-ClPh | 3-Me | H | bond | 427 |
| 72 | H | 6-MeO | H | 4-ClPh | H | H | bond | 429 |
| 73 | H | 6-MeO | H | 4-ClPh | 3-Me | H | bond | 443 |
| 155 | H | 6-MeO | H | 3-ClPh | H | H | bond | 429 |
| 156 | H | 6-MeO | H | 2,4-diClPh | H | H | bond | 464 |
| 157 | H | 6-MeO | H | 2-Me-4-ClPh | H | H | bond | 443 |
| 158 | H | 6-MeO | H | 4-MeOPh | H | H | bond | 425 |
| 159 | H | 6-MeO | H | 4-CNPh | H | H | bond | 420 |
| 160 | H | 6-MeO | H | PhO | H | H | O | 411 |
| 161 | H | 6-MeO | H | 4-ClPhO | H | H | O | 445 |
| 162 | H | 6-MeO | H | 2-thienyt | H | H | bond | 401 |
| 163 | H | 6-MeO | H | 3-thienyl | H | H | bond | 401 |
| 164 | H | 6-MeO | H | 3-furanyl | H | H | bond | 385 |
| 165 | H | 6-MeO | H | 4-pyridyl | H | H | bond | 396 |
| 166 | H | H | H | 4-ClPh | 3-F | H | bond | 417 |

All of the compounds listed below in table 3 relate to compounds of formula (IE):

TABLE 3

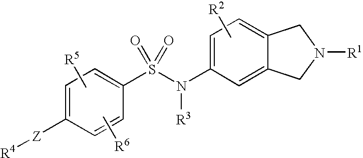

| Example | R¹ | R² | R³ | R⁴ | R⁵ | R⁶ | Z | MH⁺ |
|---|---|---|---|---|---|---|---|---|
| 9 | H | H | H | 4-ClPh | H | H | bond | 385 |
| 10 | Me | H | H | 4-ClPh | H | H | bond | 399 |
| 167 | H | H | H | 4-ClPh | 3-Me | H | bond | 399 |
| 168 | Me | H | H | 4-ClPh | 3-Me | H | bond | 413 |
| 169 | H | H | H | 4-ClPh | 3-F | H | bond | 403 |
| 170 | Me | H | H | 4-ClPh | 3-F | H | bond | 417 |
| 171 | H | H | H | 4-ClPh | 3-CF₃ | H | bond | 453 |
| 172 | H | H | H | 4-ClPh | 3-MeO | H | bond | 415 |
| 173 | Me | H | H | 4-ClPh | 3-MeO | H | bond | 429 |
| 174 | Me | H | H | 4-ClPh | 3-CF₃ | H | bond | 467 |

1. A compound of formula (I) 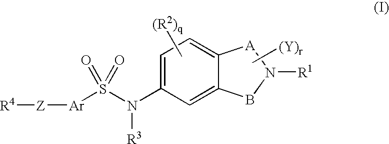
wherein
A and B represent the groups —(CH₂)ₘ- and —(CH₂)ₙ-respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆alkoxyC₁₋₆alkyl, C₃₋₇cycloalkylC₁₋₆alkoxy, —(CH₂)ₚC₃₋₆cycloalkyl, —(CH₂)ₚC₃₋₆cycloalkyloxy, —COC₁₋₆alkyl, —SO₂C₁₋₆alkyl, —SOC₁₋₆alkyl, —S—C₁₋₆alkyl, C₁₋₆alkylsulfonyloxy, C₁₋₆alkylsulfonylC₁₋₆alkyl, —CO₂C₁₋₆alkyl, —CO₂NR⁷R⁸, —SO₂NR⁷R⁸, C₁₋₆alkylsulfonamido, C₁₋₆alkylsulfonamidoC₁₋₆alkyl, —(CH₂)ₚNR⁷R⁸, C₁₋₆alkylamidoC₁₋₆alkyl, —(CH₂)ₚNR⁷COR⁸, arylsulfonyl, arylsulfonyloxy, arylsulfonylC₁₋₆alkyl, arylsulfonamido, arylcarboxamido, arylsulfonamidoC₁₋₆alkyl, arylcarboxamidoC₁₋₆alkyl, aroyl, aroylC₁₋₆alkyl, arylC₁₋₆alkanoyl, —SO₂NR⁷R⁸, optionally substituted aryl, optionally substituted heteroaryl or optionally substituted heterocyclyl, or a group CONR⁷R⁸ or SO₂NR⁷R⁸ wherein R⁷ and R⁸ together may be fused to form a 5- 7-membered aromatic or non-aromatic heterocyclic ring optionally interrupted by an O or S atom;
R³ represents hydrogen or C₁₋₆alkyl;
Ar represents optionally substituted phenyl or optionally substituted monocyclic heteroaryl group;
R⁴ represents optionally substituted aryl or optionally substituted heteroaryl;
R⁷ and R⁸ each independently represent hydrogen, C₁₋₆alkyl or together form a 5- to 7-membered heterocyclic ring;
Z represents a bond, an oxygen atom or C₁₋₆alkyl:
Y represents hydrogen or C₁₋₆alkyl;
m and n independently represent an integer selected from 1 and 2;
p independently represents an integer selected from 0, 1, 2 and 3;
q represents an integer from 1 to 3;
r represents an integer from 1 to 4;
or a pharmaceutically acceptable salt or solvate thereof.

2. A compound of formula (I) which is
4-(4-Chloro-phenyl)-N-(2,3,4,5-tetrahydro-1H-3-benzazepin-7-yl)-benzenesulfonamide;
4-(4-Chloro-phenyl)-N-(3-methyl-2,3,4,5-tetrahydro-1H-3-benzazepin-7-yl)-benzenesulfonamide;
4-(4-Chloro-phenyl)-N-methyl-N-(2,3,4,5-tetrahydro-1H-3-benzazepin-7-yl)-benzenesulfonamide;
4-(4-Chloro-phenyl)-N-methyl-N-(3-methyl-2,3,4,5-tetrahydro-1H-3-benzazepin-7-yl)-benzenesulfonamide;
4-(3,4-Dichloro-phenyl)-N-(2,3,4,5-tetrahydro-1H-3-benzazepin-7-yl)-benzenesulfonamide hydrochloride;
4-(4-Chloro-phenyl)-N-(8-methoxy-2,3,4,5-tetrahydro-1H-3-benzazepin-7-yl)-benzenesulfonamide hydrochloride;
4-(4-Chloro-phenyl)-N-(8-methoxy-3-methyl-2,3,4,5-tetrahydro-1H-3-benzazepin-7-yl)-benzenesulfonamide hydrochloride;
4-(4-Chloro-phenyl)-N-(1,2,3,4-tetrahydro-isoquinolin-7-yl)-benzenesulfonamide;
4-(4-Chloro-phenyl)-N-(2,3-dihydro-1H-isoindol-5-yl)-benzenesulfonamide hydrochloride;
4-(4-Chloro-phenyl)-N-(2-methyl-2,3-dihydro-1H-isoindol-5-yl)-benzenesulfonamide;
4-(4-Chloro-phenyl)-3-methyl-N-(2,3,4,5-tetrahydro-1H-3-benzazepin-7-yl)-benzenesulfonamide hydrochloride;
4-(4-Chloro-phenyl)-3-methyl-N-(3-methyl-2,3,4,5-tetrahydro-1H-3-benzazepin-7-yl)-benzenesulfonamide;
4-(4-Chloro-phenyl)-3-methyl-N-(8-methoxy-2,3,4,5-tetrahydro-1H-3-benzazepin-7-yl)-benzenesulfonamide hydrochloride;
4-(4-Chloro-phenyl)-3-methyl-N-(8-methoxy-3-,methyl-2,3,4,5-tetrahydro-1H-3-benzazepin-7-yl)-benzenesulfonamide;
4-(5-Chloro-thiophen-2-yl)-N-(8-methoxy-3-methyl-2,3,4,5-tetrahydro-1H-benzo[d]azepin-7-yl)-benzenesulfonamide;
4-(5-Chloro-thiophen-2-yl)-2-fluoro-N-(8-methoxy-3-methyl-2,3,4,5-tetrahydro-1H-benzo[d]azepin-7-yl)-benzenesulfonamide;
4-(4-Chloro-phenyl)-N-(8-dimethylamino-3-methyl-2,3,4,5-tetrahydro-1H-benzazepin-7-yl)-benzenesulfonamide hydrochloride and
4-(4-fluorobenzyl)-N-(3-methyl-2,3,4,5-tetrahydro-1H-benzo[d]azepin-7-yl)-benzenesulfonamide hydrochloride.

3. A pharmaceutical composition comprising a compound of formula (I) as claimed in claim 1 or a pharmaceutically acceptable salt thereof and a pharmaceutically acceptable carrier therefor.

4. A compound of formula (I) or a pharmaceutically acceptable salt or solvate thereof as claimed in claim 1, for use in therapy.

5. A compound of formula (I) or a pharmaceutically acceptable salt or solvate thereof as claimed in claim 1 for use in a condition which requires modulation of a dopamine receptor.

6. A compound of formula (I) or a pharmaceutically acceptable salt or solvate thereof according to claim 5 wherein the condition is selected from psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

7. (canceled)

8. (canceled)

9. A method of treating a condition which requires modulation of a dopamine receptor, which comprises administering to a mammal in need thereof an effective amount of a compound of formula (I) or a pharmaceutically acceptable salt or solvate thereof as claimed in claim 1.

10. A method of treating a condition according to claim 9 wherein the condition is selected from psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.
